(19) United States
(12) Patent Application Publication
(10) Pub. No.: US 2022/0079683 A1
(43) Pub. Date: Mar. 17, 2022

(54) REGISTERING OPTICAL SHAPE SENSING DEVICE WITH THREE-DIMENSIONAL REPRESENTATION OF REGION OF INTEREST

(71) Applicant: KONINKLIJKE PHILIPS N.V., EINDHOVEN (NL)

(72) Inventors: Torre Michelle BYDLON, MELROSE, MA (US); Paul THIENPHRAPA, CAMBRIDGE, MA (US); Alyssa TORJESEN, CHARLESTOWN, MA (US)

(21) Appl. No.: 15/734,023

(22) PCT Filed: Jun. 24, 2019
(86) PCT No.: PCT/EP2019/066572

§ 371 Date: Dec. 1, 2020

Related U.S. Application Data
(60) Provisional application No. 62/692,690, filed on Jun. 30, 2018.

(51) Int. Cl. A61B 34/20; G06T 7/38; G06T 7/11; A61B 90/00

(52) U.S. Cl. CPC A61B 34/20; G06T 7/38; G06T 7/11; A61B 90/37; A61B 2090/065; A61B 2034/2061; A61B 2034/2051; A61B 2090/374; A61B 2090/3764; A61B 2034/2063; G06T 2207/10028

(57) ABSTRACT

Systems and methods are provided for registering a shape sensing device, such as an optical shape sensing (OSS) device, with a previously obtained three-dimensional (3D) representation of a region of interest, the shape sensing device including an outer body for maneuvering through a passage in the region of interest and a force sensing region integrated with the outer body. The method determines multiple points at which an end of the outer body contacts a surface of an object in the region of interest, based on forces exerted on the end when contacting the surface and detected by the force sensing region; and registering the determined points with points in the 3D representation of the region of interest so that the registered points are in a common space.

### PRIORITY

The present application claims priority under 35 U.S.C. § 119(e) from commonly owned U.S. Provisional Application No. 62/665,583, filed on May 2, 2018, to Torre Bydlon et al. The entire disclosure of U.S. Provisional Application No. 62/665,583 is specifically incorporated herein by reference.

### TECHNICAL FIELD

This disclosure relates to shape and position sensing enabled devices, e.g., used for minimally invasive medical procedures using an interventional instrument, including optical shape sensing (OSS) enabled devices (OSS devices) including multicore optical fiber extending longitudinally through an outer body. Shape sensing thus occurs along the entire length of the multicore optical fiber to its distal tip. The determined device shape of portions of a region of interest may be registered to a previously obtained three-dimensional (3D) representation of the region of interest and portions of an object located therein. The registration allows the position of the OSS device to be displayed with the previously obtained 3D representation of the region of interest to visualize the relative location of the OSS device within the 3D representation without the need for additional data capture (e.g., imaging) to obtain additional 3D representations.

Other types of shape and position sensing enabled devices include electromagnetic (EM) tracking devices with one or more EM sensors, insitu tracking devices (intelligent sensing for instrument tracking using ultrasound), dielectric sensing devices, and robotic tracking devices, all of which have the potential to provide information regarding the position of the interventional instrument and/or a length of the interventional instrument. Registration of such other types of allows shape and position sensing enabled devices likewise allows the positions of these devices to be displayed with previously obtained 3D representations of the region of interest to visualize the relative location of the devices the need for additional data capture (e.g., imaging), in essentially the same manner as used for OSS enabled devices. Also, the shape information may be derived from various types of localization technology, including 1D and 2D representations, as well as the 3D representation.

### BACKGROUND

OSS-enabled devices (“OSS devices”) use light along a multicore optical fiber for device localization and navigation during surgical intervention, for example. Generally, distributed strain measurements in the optical fiber are made using characteristic Rayleigh backscatter or controlled grating patterns. The shape along the optical fiber begins at a specific point along the sensor, known as the launch or z=0. Subsequent shape position and orientation of the body of the OSS device are determined relative to that point.

The multicore optical fibers may be integrated into medical OSS devices in order to provide live guidance of the devices during minimally invasive procedures, which reduce discomfort and recovery time of a patient. The integrated, multicore optical fibers provide position and orientation information of the entire OSS device, including the shape of the OSS device. For example, an OSS device may include a shape-sensed guidewire or shape-sensed catheter, and may be used for navigation to a renal artery, with the guidance information being overlaid on a pre-operative x-ray or computer tomography (CT) image.

To properly view an OSS enabled device with respect to patient anatomy a three-dimensional (3D) to two-dimension (2D) (3D-2D) registration is performed to bring a 3D model of the vasculature, for example, into 2D x-ray imaging space. The OSS enabled device is then registered to the 2D x-ray image so that the 3D model, OSS enabled device, and the 2D x-ray image(s) all exist in the same coordinate system. However, multiple 2D x-ray images and/or 3D images or volumes need to be acquired to complete the registration. The additional radiation incurred by a patient due to the multiple x-ray images is not desirable, and could be avoided if an OSS to anatomy registration could be completed without x-ray.

### SUMMARY

According to illustrative embodiment, a method is provided for registering shape sensing enabled devices, such as an optical shape sensing (OSS) device, for example, with a previously obtained representation of a region of interest, such as a 1D, 2D or 3D representation of a region of interest. The OSS device includes an outer body, which may be elongated and flexible, for maneuvering through a passage in the region of interest and may also include a force sensing region integrated with the outer body. The method includes determining points at which a distal end of the outer body contacts a surface of an object in the region of interest, based on forces exerted on the distal end when contacting the surface and detected by the force sensing region; and associating the determined points with points in the representation of the region of interest so that the associated points are in a common space.

According to another illustrative embodiment, a method is provided for registering a shape sensing device, such as an OSS device, for example, with a previously obtained representation of a region of interest, such as a 1D, 2D or 3D representation of a region of interest. The shape sensing device includes an outer body for maneuvering through a passage in the region of interest. The method includes defining a planned path in the representation of the region of interest, the planned path substantially corresponding to the passage; inserting the shape sensing device in the passage to begin navigating the shape sensing device through the passage at an initial position; at an initial time, registering the initial position of the shape sensing device and the planned path in a shape space, and determining an initial transformation algorithm using the registration in the shape space to transform the initial position of the shape sensing device to a region space of the representation of the region of interest; at subsequent times, while continuing to navigate the shape sensing device through the passage, applying the transformation algorithm to the shape sensing device to iteratively transform subsequent positions of the shape sensing device corresponding to the subsequent times from the shape space into the region space; determining a best overlapping region between the planned path and the subsequent positions of the shape sensing device in the region space, the best overlapping region comprising a portion of the representation of the region of interest in which the subsequent positions of the shape sensing device most closely coincide with the planned path; registering the subsequent positions of the shape sensing device only in the best overlapping region and the planned path only in the best overlapping region in the space shape, and determining an updated transformation algorithm using the subsequent registration in the best overlapping region; and applying the updated transformation algorithm to the shape sensing device to again transform the subsequent positions of the shape sensing device from the shape space into the region space.

### BRIEF DESCRIPTION OF THE DRAWINGS

The present invention will be more readily understood from the detailed description of exemplary embodiments presented below considered in conjunction with the accompanying drawings, as follows.

FIG. 1 is a simplified cross-sectional diagram of an optical shape sensing device including a force sensing region, according to a representative embodiment.

FIG. 2 is a simplified schematic block diagram showing an imaging system for imaging a region of interest using an OSS sensing device capable of force detection, according to a representative embodiment.

FIG. 3 is a simplified flow diagram of a method for registering an OSS device with a 3D representation of a region of interest, according to a representative embodiment.

FIG. 4 depicts an illustrative segmented surface model of vasculature with surface points that were in contact with an OSS device, according to a representative embodiment.

FIG. 5 is a simplified flow diagram of a method for registering an OSS device with a 3D representation of a region of interest, according to a representative embodiment.

FIG. 6 is an illustrative 3D representation on a region of interest, including a planned path and an OSS device registered to the region of interest, according to a representative embodiment.

FIG. 7 is an illustrative 3D representation on a region of interest at different times, including a best overlapping region, according to a representative embodiment.

FIG. 8 is a simplified schematic diagram of a cut-away view of an optical shape sensing device including a force sensing region, according to a representative embodiment.

FIG. 9 is a simplified schematic diagram of a cut-away view of an optical shape sensing device including a force sensing region, according to a representative embodiment.

FIG. 10 is a simplified schematic diagram of a cut-away view of an optical shape sensing device including a more proximally located force sensing region, according to a representative embodiment.

FIG. 11 is a simplified schematic diagram of a cut-away view of an optical shape sensing device including a force sensing region, with no rigid tube, according to a representative embodiment.

FIG. 12 is a plan view of an optical shape sensing device including a force sensing region having a coil spring, according to a representative embodiment.

FIG. 13 is a plan view of an optical shape sensing device including a force sensing region having a coil spring, according to a representative embodiment.

FIG. 14A is a simplified cross-sectional diagram of an optical shape sensing device including a force sensing region in which multicore optical fiber has helical pattern, according to a representative embodiment.

FIG. 14B is a simplified cross-sectional diagram of an optical shape sensing device including a force sensing region in which multicore optical fiber has helical pattern, according to a representative embodiment.

FIG. 15 is a simplified cross-sectional diagram of an optical shape sensing device including a force sensing region for sensing torsion, according to a representative embodiment.

FIG. 16 is a simplified cross-sectional diagram of an optical shape sensing device including a force sensing region for sensing buckling of the optical shape sensing device, according to a representative embodiment.

FIG. 17A is a simplified transparent plan view of an optical shape sensing device including a force sensing region, according to a representative embodiment.

FIG. 17B is a simplified transparent plan view of an optical shape sensing device including a force sensing region, according to a representative embodiment.

FIG. 18 is a simplified cross-sectional diagram of an optical shape sensing device including multiple force sensing regions embedded in compliant material, according to a representative embodiment.

FIG. 19 is a simplified schematic diagram of a cut-away view of an optical shape sensing device including a force sensing region and a stopper, according to a representative embodiment.

### DETAILED DESCRIPTION

The present invention will now be described more fully hereinafter with reference to the accompanying drawings, in which embodiments of the present invention are shown. The present invention may, however, be embodied in different forms and should not be construed as limited to the embodiments set forth herein. Rather, these embodiments are provided as teaching examples of the invention.

Generally, OSS devices use light along a multicore optical fiber for device localization and navigation during surgical intervention, for example. Distributed strain measurements in the optical fiber may be made using characteristic Rayleigh backscatter or controlled grating patterns, for example. The shape along the multicore optical fiber begins at a specific point along the sensor, known as the launch or z=0. Subsequent shape position and orientation of the OSS device are determined relative to that point. The multicore optical fiber may be integrated into medical OSS devices, for example, to provide live guidance of the devices during minimally invasive procedures by providing position and orientation information of the entire OSS device, including the shape of the OSS device. Notably, the multicore optical fibers contain more information than just position and orientation of the OSS device. For example, axial strain on the optical fibers may be used to determine how much force is applied to the tip of the OSS device via the compression (or tension) of fiber Bragg gratings (FBGs), which are useful for sensing axial forces. Hence, an OSS device can be constructed in such a way that it can simultaneously determine its shape and the amount of force applied to the tip of the device

More particularly, FBGs can be used to measure axial strain in optical fiber, where axial strain is directly related to temperature changes and forces applied to the optical fiber. When temperature is decoupled from the measurement (e.g., assumed to be constant), axial strain is proportional to axial forces on the optical fiber, and can be used to measure the axial force. Also, when multiple FBGs are located along the length of an optical fiber, the shape of the optical fiber may be determined. OSS devices incorporating FBGs may be used in minimally invasive surgery. For example, OSS guidewires may be used to measure axial forces for cardiovascular procedures, such as chronic total occlusion (CTO) crossings, confirming tissue contact for ablations in the heart, transseptal puncture, and vessel wall interactions. In all of these cases it is important to know the amount of force being applied to tissue in order to prevent damage.

In multicore optical fiber with FBGs along the entire length of the optical fiber, signal losses at the distal tip of the optical fiber can obscure the FBG signals, diminishing shape sensing quality at the distal tip. A termination piece may be bound to a multicore optical fiber to improve signal quality at the distal tip of the multicore optical fiber, thereby permitting shape sensing to be performed all the way to the distal tip of the multicore optical fiber. The termination piece also enables force sensing to the distal tip of the multicore optical fiber. The term “shape sensing” used herein includes estimation, projection, and averaging of shape beyond the optical fiber, particularly with regard to projecting shape to a distal tip of the termination piece. The shape of the termination piece, or the remainder of the distal OSS device (the end of which may substantially correspond to the distal tip of the termination piece), may be determined in various ways, such as projecting the shape in a straight line from the distal tip of the multicore optical fiber to the distal tip of the termination piece.

An OSS device provides 3D (x,y,z) coordinates in a 3D coordinate system, defined in “shape space.” An x-ray image has 2D (x,y) coordinates in a 2D coordinate system, defined in “x-ray space” or “2D space;” and a CT image (or any other 3D imaging modality) has 3D (x,y,z) coordinates in a 3D coordinate system, defined as “CT space” or “region space.” A 3D model of the patient anatomy can be created from the CT (or other 3D) images in “CT space”. To properly view an OSS enabled device oriented properly with respect to patient anatomy (or the 3D model), the “shape space” and “x-ray space” need to be registered together as an intermediate coordinate system, and “x-ray space” and “CT space” need to be registered together. At least two 3D-2D registrations are performed to bring the 3D model, the OSS device and 2D x-rays all into the same coordinate system. Alternatively, any of the three coordinate systems may include fiducials that more immediately relate one coordinate system to another, such that the registration between the two is automatic to the user.

Conventionally, this is done by selecting the known tip of the OSS device in two different x-ray projection images. The problem with this is that multiple x-ray images/volumes need to be acquired to complete the registration. However, according to various embodiments, the additional radiation is avoided since OSS-anatomy registration can be completed without the x-ray. This is also advantageous for procedures that may not use intra-operative x-rays, but still require 3D guidance during the procedure, such as bronchoscopies. For these procedures, patients typically have a pre-operative acquired CT or MRI scan that can be used to generate 3D models of their anatomy. Registration methods are used to register the pre-operative 3D models (and/or 3D images/3D volumes) with medical devices used intra-operatively without intra-operative x-ray(s).

It should be understood that the disclosure is provided in terms of medical instruments; however, the present teachings are much broader and are applicable to any imaging instruments and imaging modalities. In some embodiments, the present principles are employed in tracking or analyzing complex biological or mechanical systems. In particular, the present principles are applicable to internal tracking procedures of biological systems and procedures in all areas of the body such as the lungs, gastro-intestinal tract, excretory organs, blood vessels, etc. The elements depicted in the figures may be implemented in various combinations of hardware and software and provide functions which may be combined in a single element or multiple elements.

It should be further understood that the terminology used herein is for purposes of describing particular embodiments only, and is not intended to be limiting. Any defined terms are in addition to the technical and scientific meanings of the defined terms as commonly understood and accepted in the technical field of the present teachings.

As used in the specification and appended claims, the terms “a”, “an” and “the” include both singular and plural referents, unless the context clearly dictates otherwise. Thus, for example, “a device” includes one device and plural devices. The statement that two or more parts or components are “coupled” shall mean that the parts are joined or operate together either directly or indirectly, i.e., through one or more intermediate parts or components, so long as a link occurs.

Directional terms/phrases and relative terms/phrases may be used to describe the various elements' relationships to one another, as illustrated in the accompanying drawings. These terms/phrases are intended to encompass different orientations of the device and/or elements in addition to the orientation depicted in the drawings.

A “computer-readable storage medium” encompasses any tangible storage medium which may store instructions which are executable by a processor of a computing device. The computer-readable storage medium may be referred to as a non-transitory computer-readable storage medium, to distinguish from transitory media such as transitory propagating signals. The computer-readable storage medium may also be referred to as a tangible computer-readable medium.

In some embodiments, a computer-readable storage medium may also be able to store data which is able to be accessed by the processor of the computing device. Examples of computer-readable storage media include, but are not limited to: a floppy disk, a magnetic hard disk drive, a solid state hard disk, flash memory, a USB thumb drive, Random Access Memory (RAM), Read Only Memory (ROM), an optical disk, a magneto-optical disk, and the register file of the processor. Examples of optical disks include Compact Disks (CD) and Digital Versatile Disks (DVD), for example CD-ROM, CD-RW, CD-R, DVD-ROM, DVD-RW, or DVD-R disks. The term computer readable-storage medium also refers to various types of recording media capable of being accessed by the computer device via a network or communication link. For example, data may be retrieved over a modem, over the internet, or over a local area network. References to a computer-readable storage medium should be interpreted as possibly being multiple computer-readable storage mediums. Various executable components of a program or programs may be stored in different locations. The computer-readable storage medium may for instance be multiple computer-readable storage medium within the same computer system. The computer-readable storage medium may also be computer-readable storage medium distributed amongst multiple computer systems or computing devices.

“Memory” is an example of a computer-readable storage medium. Computer memory is any memory which is directly accessible to a processor. Examples of computer memory include, but are not limited to RAM memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

Computer storage is any non-volatile computer-readable storage medium. Examples of computer storage include, but are not limited to: a hard disk drive, a USB thumb drive, a floppy drive, a smart card, a DVD, a CD-ROM, and a solid state hard drive. In some embodiments computer storage may also be computer memory or vice versa. References to “computer storage” or “storage” should be interpreted as possibly including multiple storage devices or components. For instance, the storage may include multiple storage devices within the same computer system or computing device. The storage may also include multiple storages distributed amongst multiple computer systems or computing devices.

A “processor” as used herein encompasses an electronic component which is able to execute a program or machine executable instruction. References to the computing device comprising “a processor” should be interpreted as possibly containing more than one processor or processing core. The processor may for instance be a multi-core processor. A processor may also refer to a collection of processors within a single computer system or distributed amongst multiple computer systems. The term computing device should also be interpreted to possibly refer to a collection or network of computing devices each comprising a processor or processors. Many programs have instructions performed by multiple processors that may be within the same computing device or which may even be distributed across multiple computing devices.

A “processing unit” as used herein encompasses one or more processors, computers, application specific integrated circuits (ASICs), field-programmable gate arrays (FPGAs), or combinations thereof, using software, firmware, hard-wired logic circuits, or combinations thereof. That is, a processing unit may be constructed of any combination of hardware, firmware or software architectures, and may include its own memory (e.g., nonvolatile memory), computer-readable storage medium and/or computer storage for storing executable software/firmware executable code and/or data that allows it to perform the various functions. In an embodiment, processing unit may include a central processing unit (CPU), for example, executing an operating system.

A “user interface” or “user input device” as used herein is an interface which allows a user or operator to interact with a computer or processing unit (computer system). A user interface may provide information or data to the operator and/or receive information or data from the operator. A user interface may enable input from an operator to be received by the computer and may provide output to the user from the computer. In other words, the user interface may allow an operator to control or manipulate a computer system and the interface may allow the computer system indicate the effects of the user's control or manipulation. The display of data or information on a display or a graphical user interface is an example of providing information to an operator. The receiving of data through a touch screen, keyboard, mouse, trackball, touchpad, pointing stick, graphics tablet, joystick, gamepad, webcam, headset, gear sticks, steering wheel, wired glove, wireless remote control, and accelerometer are all examples of user interface components which enable the receiving of information or data from a user.

A “hardware interface” encompasses an interface which enables the processor of a computer system to interact with and/or control an external computing device and/or apparatus. A hardware interface may allow a processor to send control signals or instructions to an external computing device and/or apparatus. A hardware interface may also enable a processor to exchange data with an external computing device and/or apparatus. Examples of a hardware interface include, but are not limited to: a universal serial bus, IEEE 1394 port, parallel port, IEEE 1284 port, serial port, RS-232 port, IEEE-488 port, Bluetooth connection, Wireless local area network connection, TCP/IP connection, Ethernet connection, control voltage interface, MIDI interface, analog input interface, and digital input interface.

A “display” or “display device” or “display unit” as used herein encompasses an output device or a user interface adapted for displaying images or data, e.g., from a computer system. A display may output visual, audio, and or tactile data. Examples of a display include, but are not limited to: a computer monitor, a television screen, a touch screen, tactile electronic display, Braille screen, Cathode ray tube (CRT), Storage tube, Bistable display, Electronic paper, Vector display, Flat panel display, Vacuum fluorescent display (VF), Light-emitting diode (LED) displays, Electroluminescent display (ELD), Plasma display panels (PDP), Liquid crystal display (LCD), Organic light-emitting diode displays (OLED), a projector, and Head-mounted display.

The embodiments herein are intended to be illustrative, and not exhaustive, such that the additional related configurations may be included. Throughout the disclosure, like numbered elements in these figures are either equivalent elements or perform the same function. Elements which have been discussed previously will not necessarily be discussed in later figures if the function is equivalent.

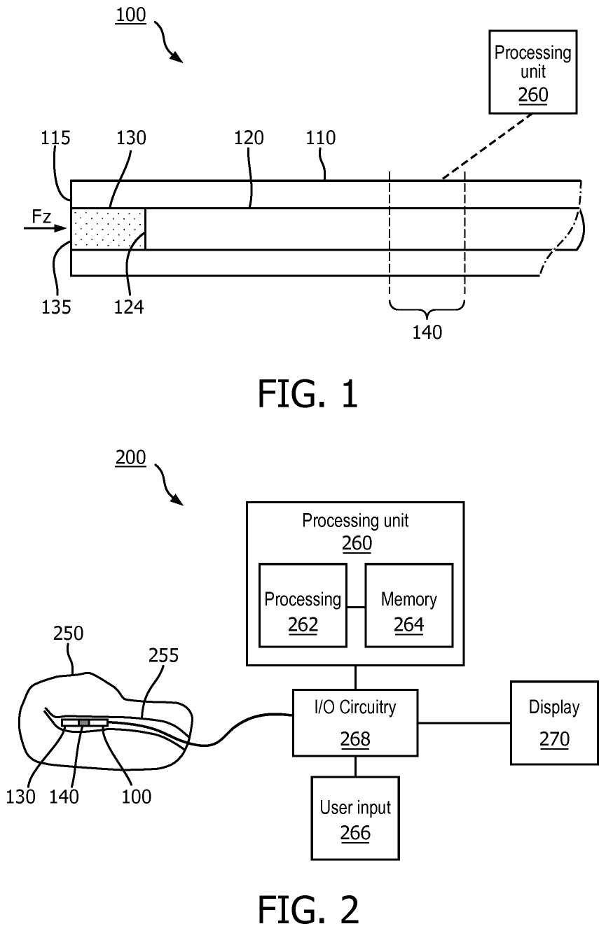

FIG. 1 is a simplified cross-sectional diagram of an optical shape sensing device including a force sensing region, according to a representative embodiment.

Referring to FIG. 1, optical shape sensing device 100 is an elongated, primarily flexible device configured for navigation through narrow passages, such as representative passage 255 in FIG. 2, although rigid portion(s) may be included for purposes of measuring axial force F_{z}, as discussed below. For example, the optical shape sensing device 100 may be configured as a shape-sensed guidewire or catheter used for navigation through vasculature of a patient during interventional medical procedures, although other configurations and/or uses may be incorporated without departing from the scope of the present teachings.

In the depicted embodiment, the optical shape sensing device 100 includes an elongated outer body 110, which includes flexible tubing, e.g., to enable maneuvering of the optical shape sensing device 100 through a passage in or around an object. The optical shape sensing device 100 also includes a multicore optical fiber 120 extending longitudinally through the elongated outer body 110, and a termination piece 130 attached to a distal tip 124 of the multicore optical fiber 120. The termination piece 130 includes a distal tip 135, which may substantially coincide with a distal end 115 of the elongated outer body 110 (as well as the distal end of the optical shape sensing device 100).

Since the termination piece 130 is bound to the mutlicore optical fiber 120, shape sensing is enabled by the optical shape sensing device 100 along the length of the multicore optical fiber 120 and to the distal tip 135 of the termination piece 130. As discussed above, this means that optical fiber shape sensing is performed to the distal tip 124 of the multicore optical fiber 120 and projected to the distal tip 135 of the termination piece 130 (collectively referred to as shape sensing). Generally, the multicore optical fiber 120 may include a central optical fiber and at least two additional optical fibers (not shown) helically wrapped around the central optical fiber, as would be apparent to one of ordinary skill in the art. The multicore optical fiber 120 enables shape sensing by tracking deformation along its length.

The optical shape sensing device 100 further includes a force sensing region 140 integrated with the elongated outer body 110. The force sensing region 140, together with a processing unit 260, discussed further below, is configured to sense an amount of axial force exerted on the distal end 115 of the elongated outer body 110. In various configurations, the amount of axial force exerted on the distal end 115 may be determined by measuring changes in axial strain on the multicore optical fiber 120 at the force sensing region 140, or by measuring torsion (twist) of the helically wrapped optical fibers of multicore optical fiber 120 at the force sensing region 140, although other types of measurements may be incorporated without departing from the scope of the present teachings. The amount of axial force exerted on the distal end 115 of the elongated outer body 110 is determined by the processing unit 260, for example, which applies the axial strain measurement and/or the torsion measurement received from the force sensing region 140 to corresponding known algorithms. Similar to the shape sensing, discussed above, the axial force exerted on the multicore optical fiber 120 is likewise detectable to the distal tip 124 of the multicore optical fiber 120.

The axial strain, in particular, measured using the multicore optical fiber 120 is directly related to temperature changes and forces applied to the multicore optical fiber 120, as mentioned above. When constant temperature is assumed, then the measured axial strain on the central optical fiber is proportional to the axial force on the distal end 115 of the elongated outer body 110. FBGs are well known to be capable of measuring forces exerted on FBG enabled devices in biological settings, for example.

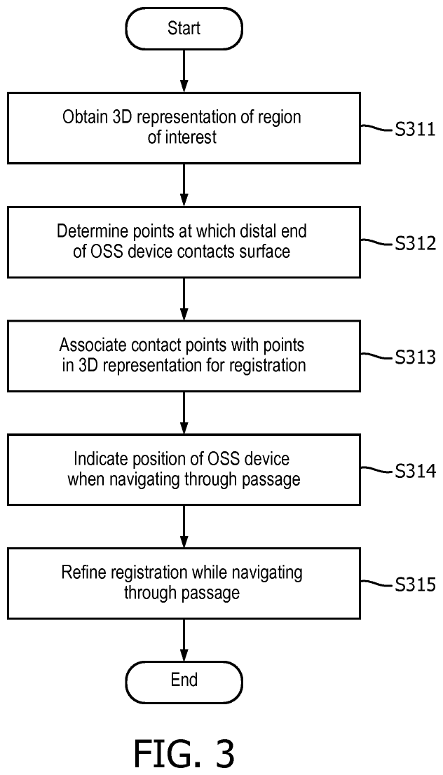

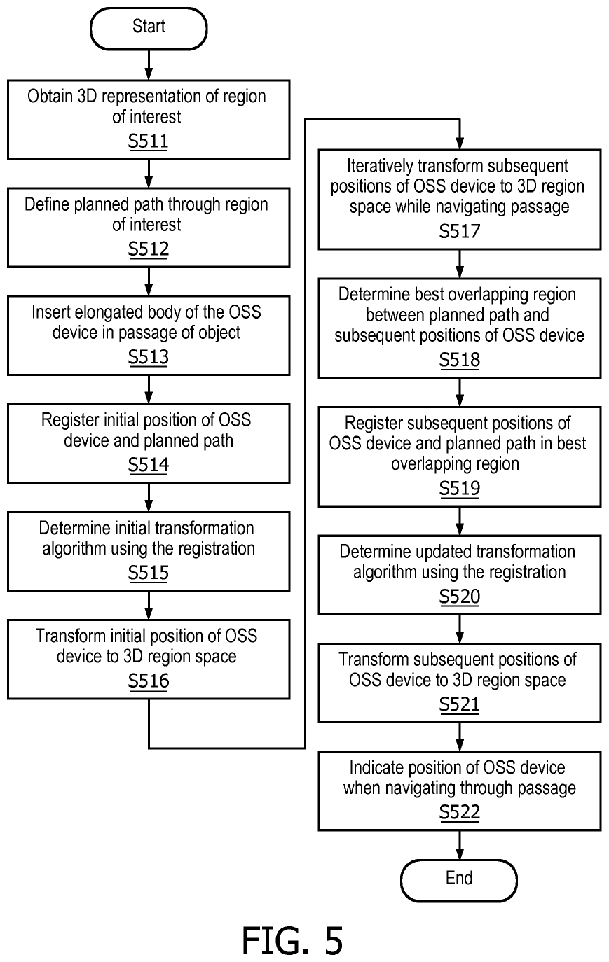

FIG. 1 shows a shape sensing device that may be used to perform the method discussed below with reference to FIG. 3. Alternative embodiments, such as the method discussed below with reference to FIG. 5, likewise may use the shape sensing device shown in FIG. 1. However, because force sensing is not needed to perform the method of FIG. 5 (although force sensing may be included in a hybrid implementation, discussed below), the shape sensing device may exclude the termination piece 130 attached to the distal tip 124 of the multicore optical fiber 120, as well as the force sensing region 140, without departing from the scope of the teachings.

FIG. 2 is a simplified schematic block diagram showing an imaging system for imaging a region of interest using an optical shape sensing device capable of force detection, according to a representative embodiment.

Referring to FIG. 2, tracking system 200 is provided for registering an OSS device with a previously obtained 3D representation of a region of interest. The tracking system 200 includes an optical shape sensing device 100 acting as an interventional instrument, such as a catheter and/or a guidewire, as discussed above with reference to FIG. 1. The optical shape sensing device 100 is insertable within a passage 255 in a region of interest 256, which includes at least a portion of an object 250 (e.g., the heart) of a subject (e.g., patient) in order to provide data to enable identification of one or more surfaces of the object 250 in the region of interest 256. In the depicted example, the passage 255 passes through the object 250, e.g., such as a coronary artery entering the heart, and thus the surface of the object 250 being identified may be an inner surface of the passage 255. Alternatively, the passage 255 may be adjacent to the object 250, and thus the surface of the object 250 being identified may be an outer surface of the object 250.

The processing unit 260 of the tracking system 200 determines points at which a distal end of the tip 130 contacts the surface of the object 250 in the region of interest 256. The points of contact may be determined based on forces exerted on the distal end of the tip 130 when contacting the surface and detected by the force sensing region 140. The processing unit 260 further associates the determined points with points in a 3D representation of the region of interest 256 so that the associated points are in a common space.

The tracking system 200 further includes the processing unit 260, a display 270, a user input 266, and input/output circuit 268. The processing unit 260 includes at least one processor 262 and at least one memory 264. The memory 264 is a non-transitory storage medium, such as Random Access Memory (RAM), Read Only Memory (ROM), a magnetic disk and/or a solid state memory, for example. The memory 264 includes instructions that, when executed by the processor 262, cause the processor 262 to receive and process the force data captured by the optical shape sensing device 100. Generally, the processing includes performing or causing to be performed one or more of the steps depicted in FIGS. 3 and 5, discussed below. For example, the processing may include identifying points of contact between the elongated body 110 of the shape sensing device 100 and surface(s) of an object 250 in the region of interest 256, determining positions of the elongated body 110, registering the points of contact and/or positions of the elongated body 110 with previously obtained 3D representations of the region of interest. The 3D representations of the region of interest may include images of the region of interest 256, such as x-ray images, CT images, MR images, cone beam CT (CBCT) images, positron emission tomography (PET) scan images, ultrasound images, optical images, and the like.

Various aspects of the process may be displayed on the display 270 (e.g., an LCD display, an OLED display, and/or a touch sensitive screen), without departing from the scope of the present teachings. For example, the display 270 may show the 3D representation of the region of interest, depictions of the OSS device 100 registered with the 3D representation of the region of interest, planned paths corresponding to the passage 255, real-time progress of the OSS device 100 through the passage 255 in the 3D representation, etc., all of which are discussed below. The user (e.g., surgeon) is able to control the display 270 and the processing unit 260 via the user input 226 (e.g., a mouse, a keyboard, a trackball, and/or a touch sensitive screen). For example, the user may isolate portions of the displayed image, zoom in and out, and the like. The optical shape sensing device 100 provides data that may be displayed, including but not limited to points of contact with a surface of the object 250, corresponding positions of the contact points on a surface of the 3D representation, and magnitudes of axial and/or longitudinal forces on the elongated outer body 110, for example. The user may pause at various positions, and move forward and backward in the displayed image as desired. The user may also control positioning of the optical shape sensing device 100 and/or create the planned path in the region of interest 256 via the user input 226.

FIG. 3 is a simplified flow diagram of a method for registering an OSS device used for an interventional procedure with a 3D representation of a region of interest, according to a representative embodiment. More particularly, FIG. 3 shows a method including OSS device to surface registration, where the surface is defined by an object in the region of interest. The OSS device (e.g., optical shape sensing device 100) includes an elongated outer body (e.g., elongated outer body 110) for maneuvering through a passage in the region of interest and a force sensing region (e.g., force sensing region 140) integrated with the elongated outer body. The various steps in FIG. 3 may be performed and/or controlled by the processing unit 260, discussed above, for example.

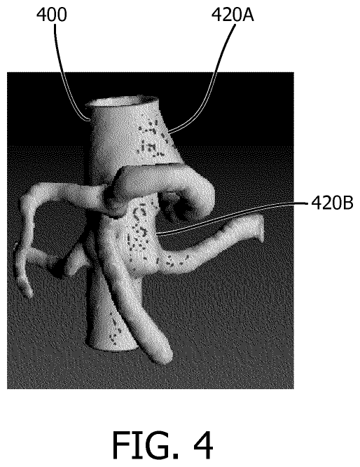

Referring to FIG. 3, a 3D representation of the region of interest is obtained in block S311. The 3D representation of the region of interest includes a 3D representation of the object (or portion of the object) located within the region of interest. The 3D representation of the region of interest may be a 3D model or image (e.g., volume), for example. The 3D model may be a segmented surface model, for example, created using multiple CT scans. Segmenting the image dataset may be done in several ways, including but not limited to, thresholding or clustering the intensity values, edge detection algorithms, region growing, model based methods, or contour based methods. Of course, other segmentation method may be incorporated without departing from the scope of the present teachings. FIG. 4 shows an example of a segmented surface model of vasculature, discussed below. The 3D image may be obtained using various imaging technologies, such as x-ray imaging, CT imaging, MR imaging, CBCT imaging, PET scan imaging, ultrasound imaging or optical imaging, for example.

In various embodiments, the 3D representation of the region of interest is acquired prior to the interventional procedure (e.g., pre-operatively), thereby avoiding the need for real-time imaging of the region of interest during the interventional procedure. This approach reduces exposure of the subject to potentially harmful imaging side effects, such as increased exposure to radiation from multiple x-ray imaging procedures, for example. That is, the additional radiation is avoided because the OSS device to 3D representation registration is completed without an x-ray (other than a pre-operative x-ray image) or other imaging while navigating the OSS device. This approach also enables the interventional procedure to take place at a location away from potentially cumbersome imaging devices and/or procedures required for various technologies, such as placing the subject in a scanner to obtain MR images. However, the 3D representation of the region of interest may be acquired during the interventional procedure, for registering with the OSS device, without departing from the scope of the present teachings.

In other embodiments, the 3D representation of the region of interest may include recognizing a known signature that affects shape data of the passage in the region of interest. When using a known signature that affects shape data, the registration of the OSS device with the 3D representation of the region of interest can be initialized without an x-ray or other image. The known signature may include a thermal signature and/or a defined curvature signature, for example. The thermal signature may be obtained by looking at a specific shape or intensity in the axial strain from the OSS device. The defined curvature signature may be obtained by looking for a specific shape or intensity in the amount of curvature calculated from the OSS device. Alternatively, the known signature may include a profile of navigation signatures derived from at least one previous procedure involving OSS navigation of the passage in the region of interest.

In block S312, multiple points (a point cloud) are determined at which a distal end of the elongated outer body contacts a surface of an object in the region of interest. The surface of the object may be an inner surface of the passage itself, or it may be another surface of the object to which the passage is adjacent, for example. The points are determined based on forces exerted on the distal end when the distal end contacts the surface and detected by the force sensing region. More particularly, the forces may be exerted on a distal tip (e.g., distal tip 135) of a termination piece (e.g., termination piece 130 attached to multicore optical fiber (e.g., multicore optical fiber 120) within the elongated body. For example, the amount of force exerted on the distal end of the elongated outer body may be determined by measuring changes in strain on the multicore optical fiber at the force sensing region of the OSS device, or by measuring torsion (twist) of the helically wrapped optical fibers of multicore optical fiber at the force sensing region. Knowing the position of the distal end of the elongated body throughout the procedure, the contact points and corresponding positions of the OSS device can be collected when a force sensing algorithm, employed in response to force detected at the force sensing region, indicates that the distal end is in contact with the surface (e.g., tissue).

In an embodiment, the forces detected at the force sensing region and used to determine points of contact between the elongated outer body and the surface of the object are axial forces. However, lateral forces and/or a combination of axial and lateral forces may be detected and used for determining point of contact, without departing from the scope of the present teachings. Lateral forces may be detected at various locations along the OSS device, and not necessarily just at the distal end, as would be apparent to one skilled in the art.

In block S313, the determined contact points are registered (or associated) with points in the 3D representation of the region of interest (e.g., the 3D model or 3D image/volume), so that the registered points are in a common space. That is, the points at which the OSS device contacts the object surface are in shape space, and the points in the 3D representation of the region of interest are in region space (or “anatomy space”). In an embodiment, registering the determined contact points with points in the 3D representation includes determining sets of 3D coordinates of each of the determined contact points in the shape space (OSSuvw), which are collected from the surface of the object, and then reregistering these sets of 3D coordinates to the associated points in the 3D representation of the region of interest to obtain the determined contact points in the region space (OSSxyz). Registering the sets of 3D coordinates to the points in the 3D representation may be accomplished using a registration algorithm, such as a deformable Iterative Closest Point (ICP) algorithm, coherent point drift, and/or robust point matching, for example.

FIG. 4 depicts an illustrative segmented surface model of vasculature with surface points that were in contact with an OSS device, according to a representative embodiment. Referring to FIG. 4, segmented surface model 400 is a 3D model of an object (vascular segment) in the region of interest. Points on an inner surface of the surface model 400, indicated by representative points 420A and 420B, are identified with OSS force sensing capabilities, as discussed above. FIG. 4 shows the contact points and the segmented surface model both registered in the region space.

Referring again to FIG. 3, in block S314, a position of the OSS device may be visually indicated on a display (e.g., on the display 270) when navigating the elongated outer body through the passage using the associated points in the common space. As mentioned above, indication of the position of the OSS device may be made in real-time, without additional imaging. Also, registration may optionally be refined in block S315 during navigation of the elongated outer body through the passage as new contact points between the elongated outer body and the surface of the object are collected. For example, new contact points may be collected as the device is being navigated, and added to the previously acquired contact points. The same registration algorithm can be applied but with the additional points. Alternatively, just the newly collected contact points may be used to register to the 3D model. Refining the registration while navigating the passage helps account for deformation between the region of interest and a previously obtained 3D representation of the region of interest (e.g., a preoperative image).

Notably, the force sensing region is capable of distinguishing among tissue stiffness at different points at which the elongated outer body contacts the surface, since stiffer or firmer tissue will cause application of a greater amount of force on the elongated outer body than softer tissue. The sensed stiffness of the contact points may be taken into account in performing registration. That is, the method of registration may further include determining stiffness of the passage at the multiple points at which the distal end of the elongated outer body contacts the surface of the object, e.g., based on axial forces exerted on the distal end. In this case, associating the determined points with points in the 3D representation of the region may include incorporating indications of stiffness for each the associated points in the common space. By incorporating determination of stiffness, differences in stiffness may be used to distinguish healthy tissue and diseased tissue, for example, where the diseased tissue exhibits greater stiffness (firmness) than the healthy tissue. Differences in the determined stiffness may be spatially associated with corresponding parts of the model.

FIG. 5 is a simplified flow diagram of a method for registering an OSS device with a 3D representation of a region of interest, according to a representative embodiment. More particularly, FIG. 5 shows a method including OSS device to path registration, where the path is planned path with respect to an object in the region of interest. The OSS device (e.g., optical shape sensing device 100) includes an elongated outer body (e.g., elongated outer body 110) for maneuvering through a passage in the region of interest, although the force sensing features (e.g., force sensing region 140, termination piece 130) are not needed. The various steps in FIG. 5 may be performed and/or controlled by the processing unit 260, for example.

Referring to FIG. 5, a 3D representation of the region of interest is obtained in block S511. The 3D representation of the region of interest includes a 3D representation of the object (or portion of the object) located within the region of interest. The 3D representation of the region of interest may be a 3D model or 3D image, for example, as discussed above. In various embodiments, the 3D representation of the region of interest is acquired prior to the interventional procedure (e.g., pre-operatively), thereby avoiding the need for real-time imaging of the region of interest during the interventional procedure. This approach reduces exposure of the subject to potentially harmful imaging side effects, and enables the interventional procedure to take place at a location away from potentially cumbersome imaging devices and/or procedures, as discussed above. In other embodiments, the 3D representation of the region of interest may include recognizing a known signature that affects shape data of the passage in the region of interest, such as a thermal signature and/or a defined curvature signature, for example.

In block S512, a planned path is defined in the 3D representation of the region of interest. The planned path substantially corresponds to the passage in the region of interest through which the elongated outer body is to be maneuvered. The planned path may be defined by the user using a previously obtained 3D representation of the region of interest, or the planned path it may be determined automatically, e.g., by processing unit 260. Defining the planned path includes forming a line, made up of multiple 3D points, through the lumen (e.g., passage 255) of the 3D representation of the region of interest. The line may be at the exact center of the lumen or it may be non-centered.

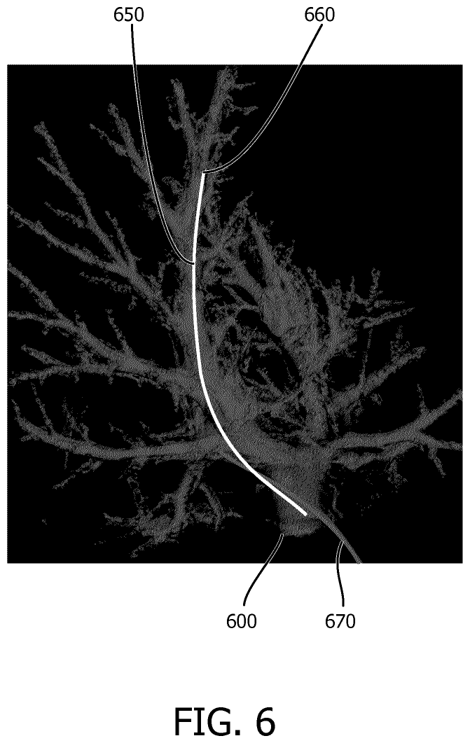

FIG. 6 is an illustrative 3D representation on a region of interest, including a planned path and an OSS device registered to the region of interest, according to a representative embodiment. Referring to FIG. 6, the 3D representation of the region of interest includes a 3D model 600 of an object (airways) in the region of interest. A planned path 620 of the OSS device is shown in the airways, which the elongated outer body 630 of the OSS device (e.g., such as the elongated outer body 110 of the shape sensing device 100) is intended to follow to reach a target location 640 at the outer branches of the airways.

In block S513, the elongated outer body of the OSS device is inserted in the passage to begin navigating the OSS device through the passage at an initial position. At an initial time (T0), the initial position of the OSS device and the planned path are registered in 3D shape space (OSSuvw) in block S514, and an initial transformation algorithm (or registration algorithm) is determined using the registration in the 3D shape space in block S515. The transformation algorithm includes how much the coordinates should be translated in x,y,z directions and how much rotation occurs around the x,y,z axes. Thus, the transformation algorithm defines translation and rotation amounts needed to associate the respective points in the same coordinate system. The initial transformation may be approximated from past procedures, for example; it need not be precise because it is updated as the OSS device is navigated through the passage.

The initial registration corresponding to the initial time T0 may be done automatically, for example, by recognizing some known signature that affects the shape data of the OSS device, such as a thermal signature or a defined curvature signature. These signatures may be derived from the anatomy itself, for example where the thermal signature may indicate the OSS device has entered the body and a difference is seen between body temperature and room temperature; or the signature may be induced mechanically. After the distal end of the elongated outer body of the OSS device experiences change according to the signature, the registration may be started. Alternatively, the initial registration may be initiated by input from the user, such as a button click, voice command, or the like. In block S516, the initial transformation algorithm is applied to the 3D shape space coordinates of the OSS device to transform the initial position of the OSS device to a 3D region space of the 3D representation of the region of interest (OSSxyz).

At subsequent times (T1, T2, T3 . . . ), while continuing to navigate the elongated outer body of the OSS device through the passage, the transformation algorithm determined in block S515 is applied to the OSS device in block S517 to iteratively transform subsequent positions of the OSS device, corresponding to the subsequent times, from the 3D shape space (OSSuvw) into the 3D region space (OSSxyz). This continuous transformation may apply to OSS devices moving in a forward path (i.e. going deeper into the body) or in a reverse path (i.e. backing up with the OSS device in the opposite direction).

In block S518, a best overlapping region (e.g., closest match) between the planned path and the detected subsequent positions of the OSS device in the 3D region space is determined at each subsequent time. The best overlapping region includes a portion of the 3D representation of the region of interest in which the subsequent positions of the OSS device most closely coincide with the planned path. For example, the best overlapping region may include the portion of the 3D representation of the region of interest in which the subsequent positions of the OSS device deviate from the planned path by less than a threshold on the distance between the points. The threshold may be pre-defined in the algorithm or it may be calculated dynamically and continuously updated based on additional information, such as how much of the device is within the body or from the anatomical context.

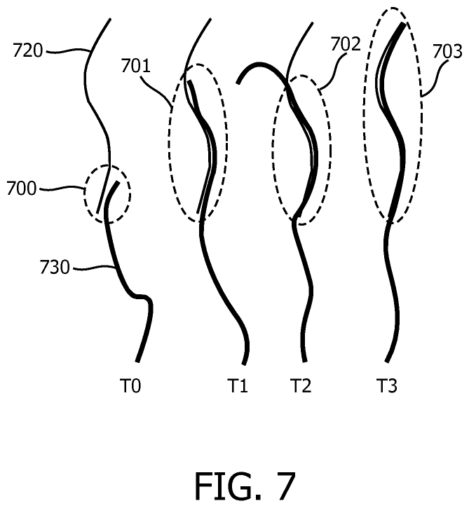

FIG. 7 is an illustrative 3D representation on a region of interest at different times, including a best overlapping region, according to a representative embodiment. Referring to FIG. 7, time T0 indicates the initial time at which the initial position of the OSS device 730 and the planned path 720 are first registered in 3D shape space. At time T0, best overlapping region 700, indicated by a dashed oval, is the region in which the position of the OSS device most closely aligns with the predetermined position of the planned path 720. As the elongated outer body 730 of the OSS device (e.g., such as the elongated outer body 110 of the shape sensing device 100) is navigated through the passage (which the planned path is intended to generally follow, as discussed above), the positions of the elongated outer body 730 are shown at subsequent predetermined times T1, T2 and T3, respectively. The best overlapping regions 701, 702 and 703 corresponding to the subsequent times T1, T2 and T3 are indicated by dashed ovals, respectively. The best overlapping regions 701, 702 and 703 are the regions in which the position of the elongated outer body 730 of the OSS device most closely aligns with the predetermined position of the planned path 720 at the subsequent times T1, T2 and T3. At the time T3, the elongated outer body 730 extends furthest into the passage, and the planned path 720 accurately represents the passage, and thus the best overlapping region 703 is largest at the time T3 compared to the other times. In an embodiment, the best overlapping region at each of the times T0, T1, T2 and T3 is determined by calculating the distance between the planned path and the OSS device and setting a defined threshold on the maximum distance, as one example. Other techniques for determining the best overlapping region may be incorporated without departing from the scope of the present teachings.

In block S519, the subsequent positions of the OSS device and the planned path, only in the best overlapping region, are registered in the 3D space shape. An updated transformation algorithm is determined in block S520 using the subsequent registration in the best overlapping region. The updated transformation algorithm is applied in block S521 to the OSS device to again transform the subsequent positions of the OSS device from the 3D shape space (OSSuvw) into the 3D region space (OSSxyz). In block S522, a position of the OSS device may be visually indicated on a display (e.g., on the display 270) when navigating the elongated outer body of the OSS device through the passage using the registered points. As mentioned above, indication of the position of the OSS device may be made in real-time, without additional imaging.

A threshold (static or dynamic) may be utilized to determine when to stop iteratively updating the registration and to simply use a previously saved transformation matrix. For example, if the device is sufficiently within the body and the registration of the OSS device to the 3D anatomical model is acceptable then registration should cease and the OSS device coordinates of future positons should be transformed based on the last saved transformation matrix. Alternatively, this stopping point may also be based on input from the user and not automatically.

It may be desirable to have an additional algorithm which monitors the likelihood that the registration is ‘good’. For example, if the registration algorithm transforms the OSS device coordinates (OSSuvw) to a location in 3D region space (OSSxyz) that is physically impossible, like the device would be outside of the body, this likelihood algorithm should trigger an error flag indicating a ‘bad’ registration. With this information the registration algorithm may be stopped automatically or the user may be required to step in. After an error flag, the registration algorithm may be re-initiated.

Notably, the OSS device may include a force sensing region (e.g., force sensing region 140) and a termination piece (e.g., termination piece 130) on the elongated outer body (e.g., elongated outer body 110), as described above. Because the ideal planned path goes through the center of the passage, such as an anatomical cavity, information about tissue contact from the force sensing region of the OSS device may be used to reduce weights of the contact positions when computing the registration. This is because the contact points tend to be away from the centerline of the planned path, if the centerline is used to define the path. Further, because positions are constantly tracked during navigation of the OSS device, the distance of the OSS device from the last contact point may be inferred, and thus the distance of the OSS device from the surface may be estimated. This enables determination of how close the OSS device currently is to the centerline of the planned path, and this determination may be used to weight each point in the point cloud to improve registration accuracy. The determining of contact points, computing of registration, the weighting of various points, the determining of distance to the centerline, and the inferring/estimating of distance of the OSS device to the surface may be all performed or in part by the processing unit 260, for example.

In various embodiments, in addition to or instead of utilizing a planned path to register the OSS device to the region (anatomy), the centerline of the passage and/or each branch of the passage (e.g., a vascular or bronchial tree) may be used as a unique shape signature for registration. The centerline of each passage and/or branch may be determined via automatic or manual segmentation of the predetermined 3D representation (e.g., preoperative image), and the OSS device can then be iteratively registered to the centerline trace, generally according to the method shown in FIG. 5, where planned path is replaced by centerline.

Also, various embodiments provide a hybrid registration, in which the elongated outer body of the OSS device includes a force sensing region. The hybrid registration includes both registration using surface contact points identified by the force sensing region (device to surface registration) and registration using the planned path (device to path registration). Simultaneous use of these methods produces quicker and more accurate registration results. For example, when the force sensing region senses contact, the OSS device is known to be in contact with a point on the surface of the object in the 3D representation of the region of interest, and that contact point will be registered against model surfaces, as discussed with reference to the method of FIG. 3. When the force sensing region does not sense contact with regard to a device point, the OSS device is known to be in a passage (or lumen) without touching the surfaces, and that device point will be registered against the planned path, or other paths within the anatomy that are not necessarily part of the surgical plan, as discussed with reference to the method of FIG. 5. Further, when the force sensing region does not sense contact, a distance from the elongated outer body to the surface may be estimated based on tracking history, and the points can be weighted as part of the deformable ICP algorithm.

For planned path registration, OSS tracking is beneficial to the extent it provides information to determine the shape the entire OSS device in a single snapshot. However, in alternative embodiments, other, single-point technologies may be used for determining the shape and/or location of an interventional device having no OSS capability, without departing from the scope of the present teachings. Examples of alternative technologies include electromagnetic (EM) tracking using one or more EM sensors, insitu tracking (intelligent sensing for instrument tracking using ultrasound), dielectric sensing, and robotic device tracking, all of which have the potential to provide information regarding the position of the distal end (tip) of the interventional device and/or a length of the interventional device. In these embodiments, the distal end of the interventional device is tracked, and the resulting trajectory is accumulated over time to identify an actual path of the interventional device, which may be displayed along with the planned path, for example. For example, in EM tracking, an EM sensor may be attached to the distal end of the interventional device, and tracking data may be collected from the EM sensor by EM sensing receiver coils while the interventional device is navigated through a passage in the region of interest. These alternative technologies may also include multiple sensors thereby enabling an alternative form of shape sensing, e.g., of the elongated outer body.

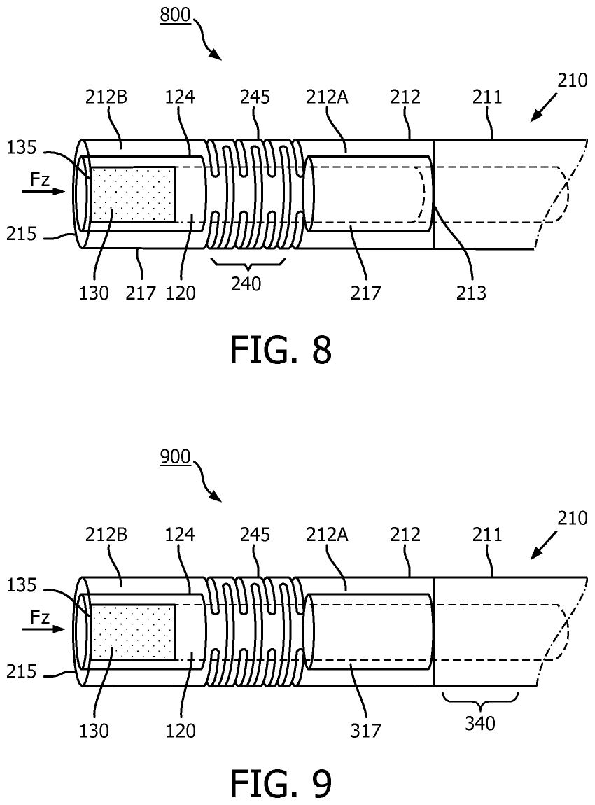

Notably, FIG. 1 shows one general example of an optical shape sensing device including a force sensing region. It is understood that other variations of optical shape sensing devices that include force sensing regions may be incorporated without departing from the scope of the present teachings. For example, FIGS. 8 and 9 are simplified schematic diagrams of a cut-away view of optical shape sensing devices including a force sensing region, respectively, according to representative embodiments.

Referring to FIG. 8, optical shape sensing device 800 includes an elongated outer body 210, which includes flexible tubing 211 and rigid tube 212 attached to the flexible tubing 211. In the depicted embodiment, the rigid tube 212 is attached to a distal end 213 of the flexible tubing 211. The flexible tubing 211 enables the maneuvering of the optical shape sensing device 200 through a passage, as discussed above. The flexible tubing 211 may be formed of various flexible materials, such as polyethylene, polyether ether ketone, polypropylene, nylon, polyimide, acetal or acrylonitrile butadiene styrene, and the rigid tube 212 may be formed of various less flexible materials, such as nitinol, stainless steel, titanium, aluminum, and various metal or plastics, such as polyether ether ketone, polypropylene, nylon, polyimide, acetal, and acrylonitrile butadiene styrene, although different materials may be incorporated without departing from the scope of the present teachings.

The optical shape sensing device 800 also includes multicore optical fiber 120 extending longitudinally through the elongated outer body 210, and a termination piece 130 attached to a distal tip 124 of the multicore optical fiber 120, as discussed above. The termination piece 130 is positioned within the rigid tube 212, and includes the distal tip 135, which may substantially coincide with a distal end 215 of the elongated outer body 210. Shape sensing is enabled by the optical shape sensing device 800 along the multicore optical fiber 120 to the distal tip 135 of the termination piece 130.

The optical shape sensing device 800 further includes a force sensing region 240 integrated with the elongated outer body 210. For example, the rigid tube 212 may be micromachined to have a proximal rigid section 212A, a distal rigid section 212B, and a middle elastic segment 245 located in between. Thus, the elastic segment 245 is located proximally from the termination piece 130. In the depicted embodiment, the force sensing region 240 of the optical shape sensing device 200 coincides with the elastic segment 245. The elastic segment 245 enables axial compression and expansion of the rigid tube 212 of the elongated outer body 210 responsive to an axial force F_{z} exerted on the distal end 215 of the elongated body 210.

Adhesive 217 binds the multicore optical fiber 120 to an inner surface of both the proximal rigid section 212A of the rigid tube 212 (at a proximal side of the elastic segment 245), and the distal rigid section 212B of the rigid tube 212 (at a distal side of the elastic segment 245). The adhesive 217 also binds the multicore optical fiber 120 to an inner surface of the termination piece 130 in the distal rigid section 212B. The adhesive 217 may be an epoxy or an anaerobic adhesive material, for example, although different materials may be incorporated without departing from the scope of the present teachings.

The design of the elastic segment 245 dictates the degree to which the optical shape sensing device 800 compresses or bends. In the depicted embodiment the elastic segment 245 comprises a pattern of slits formed around an outer circumference of the rigid tube 212. The pattern of slits may be formed in the rigid tube 212 by 3D printing, laser cutting, micro-machining, casting, or lithographic techniques, for example, although other slit formation techniques may be incorporated without departing from the scope of the present teachings. Also, the pattern of slits may be formed prior to attachment of the rigid tube 212 to the flexible tubing 211. In alternative embodiments, the elastic segment 245 may comprise other types of flexible structures, such as a laser cut design (not shown) formed around the outer circumference of the rigid tube 212, or a coil spring, for example.

The force sensing region 240, together with the processing unit 260 (not shown in FIG. 8), is configured to sense the amount of axial force exerted on the distal end 215 of the elongated outer body 210, which corresponds to the distal end of the rigid tube 212. When the elastic segment 245 compresses, the bare (without adhesive 217) multicore optical fiber 120 between the proximal and distal rigid sections 212A and 212B also compresses, and the axial strain in this area is used to calculate the applied force. Determination of the amount of axial force exerted on the distal end 215 involves measuring changes in axial strain on the central optical fiber of the multicore optical fiber 120 at the force sensing region 240, as discussed above.

FIG. 9 is a simplified schematic diagram of a cut-away view of an optical shape sensing device including a force sensing region, according to a representative embodiment. Referring to FIG. 9, optical shape sensing device 900 is substantially the same as the optical shape sensing device 800, except that a force sensing region 340 is located in a portion of the flexible tubing 211 immediately adjacent to a proximal end of the rigid tube 212, next to the proximal rigid section 212A, as opposed to coinciding with the elastic segment 245.

That is, the optical shape sensing device 900 includes the elongated outer body 210, which includes the flexible tubing 211 and the rigid tube 212 attached to the flexible tubing 211. The optical shape sensing device 900 also includes the multicore optical fiber 120 extending through the elongated outer body 210, and a termination piece 130 attached to a distal tip 124 of the multicore optical fiber 120, as discussed above, and positioned within the rigid tube 212. As in the previous embodiment, shape sensing is enabled by the optical shape sensing device 900 along the multicore optical fiber 120 to the distal tip 135 of the termination piece 130.

The optical shape sensing device 900 further includes the elastic segment 245 located in the rigid tube 212 proximally from the termination piece 130. Adhesive 317 binds the multicore optical fiber 120 to the inner surface of the proximal rigid section 212A of the rigid tube 212, but not to the distal rigid section 212B. Accordingly, the multicore optical fiber 120 and the termination piece 130 are free to float within the distal rigid segment 212B and the elastic segment 245. Any compression (and axial strain) of the multicore optical fiber 120 responsive to an axial force F_{z} exerted on the distal end 215 of the elongated body 210 would therefore occur just proximally to the proximal rigid section 212A of the rigid tube 212, which is fixed to the multicore optical fiber 120 by the adhesive 317. This compression (and axial strain) would be sensed through the force sensing region 340. Determination of the amount of axial force exerted on the distal end 215 involves measuring changes in the axial strain on the central optical fiber of the multicore optical fiber 120 at the force sensing region 340, as discussed above. The adhesive 317 may be an epoxy or an anaerobic adhesive material, for example, although different materials may be incorporated without departing from the scope of the present teachings.

Other embodiments of optical shape sensing devices that include force sensing regions, discussed below, may be implemented. These embodiments may be used for OSS device tracking and force detection, as described herein, without departing from the scope of the present teachings.

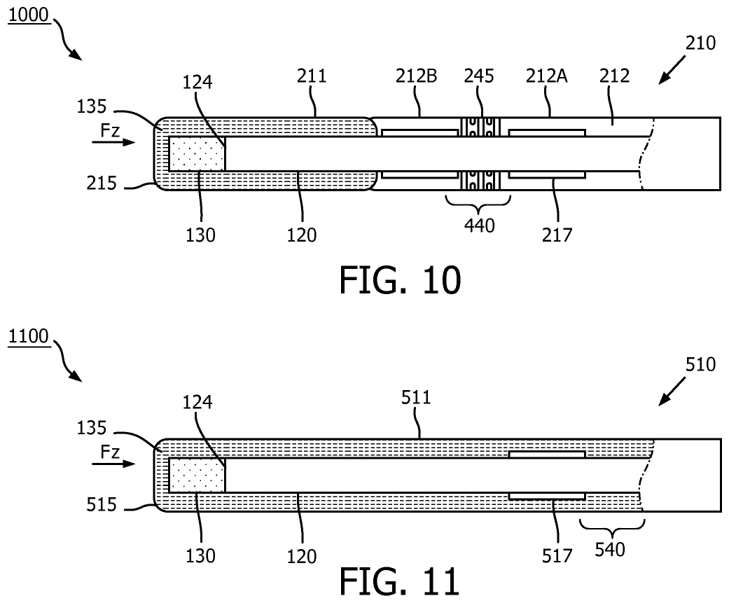

FIG. 10 is a simplified schematic diagram of a cut-away view of an optical shape sensing device including a more proximally located force sensing region, according to a representative embodiment. Referring to FIG. 10, optical shape sensing device 1000 is substantially the same as the optical shape sensing device 800, except that the relative locations of the flexible tubing 211 and the rigid tube 212 are reversed, with additional flexible tubing (not shown) on the proximal end of the rigid tube 212, enabling the flexibility for navigation through passages. The elastic segment 245 is located between the proximal rigid section 212A and the distal rigid section 212B of the rigid tube 212, and a force sensing region 440 of the optical shape sensing device 1000 coincides with the elastic segment 245.

That is, the optical shape sensing device 1000 includes the elongated outer body 210′, which includes the flexible tubing 211 and the rigid tube 212 attached to the flexible tubing 211 at a proximal end 216 of the flexible tubing 211 (as opposed to being attached to the distal end 213). The optical shape sensing device 1000 also includes the multicore optical fiber 120 extending through the elongated outer body 210′, and a termination piece 130 attached to a distal tip 124 of the multicore optical fiber 120, as discussed above. The termination piece 130 is positioned within the flexible tubing 211. Shape sensing is enabled by the optical shape sensing device 1000 along the multicore optical fiber 120 to the distal tip 135 of the termination piece 130.

The optical shape sensing device 1000 further includes the elastic segment 245 located in the rigid tube 212 proximally from the termination piece 130 and the flexible tubing 211. Adhesive 217 binds the multicore optical fiber 120 to an inner surface of both the proximal rigid section 212A of the rigid tube 212, and the distal rigid section 212B of the rigid tube 212. The elastic segment 245 enables axial compression and expansion of the rigid tube 212 of the elongated outer body 210′ responsive to an axial force F_{z} exerted on the distal end 215 of the elongated body 210. No adhesive binds the termination piece 130 to the flexible tubing 211. Accordingly, the multicore optical fiber 120 and the termination piece 130 are free to float within the flexible tubing 211 and the elastic segment 245. Any compression (and axial strain) of the multicore optical fiber 120 responsive to an axial force F_{z} exerted on the distal end 215 of the elongated outer body 210 would therefore occur in the elastic segment 245. Determination of the amount of axial force exerted on the distal end 215 involves measuring changes in the axial strain on the central optical fiber of the multicore optical fiber 120 at the force sensing region 340, as discussed above.

FIG. 11 is a simplified schematic diagram of a cut-away view of an optical shape sensing device including a force sensing region, according to a representative embodiment. Referring to FIG. 11, optical shape sensing device 1100 does not include a rigid tube, such rigid tube 212. The multicore optical fiber 120 therefore extends entirely through flexible tubing (flexible tubing 511). A force sensing region 540 of the optical shape sensing device 1100 is located proximally to a section of adhesive 217 between the multicore optical fiber 120 and the inner surface of the flexible tubing 511.

More particularly, the optical shape sensing device 1100 includes an elongated outer body 510, which includes the flexible tubing 511. The multicore optical fiber 120 extends through the flexible tubing 511, and a termination piece 130 is attached to a distal tip 124 of the multicore optical fiber 120, as discussed above. The termination piece 130 is also located within the flexible tubing 511. Shape sensing is enabled by the optical shape sensing device 1100 along the multicore optical fiber 120 to the distal tip 135 of the termination piece 130.

Adhesive 517 binds the multicore optical fiber 120 to the inner surface of the flexible tubing 511 proximally from the termination piece 130. In the depicted embodiment, the adhesive 517 is not immediately adjacent to the termination piece 130, but rather is located a distance from the termination piece 130, which is sufficient to allow some floating of the multicore optical fiber 120 before the location of the adhesive 517. In other words, the multicore optical fiber 120 and the termination piece 130 are free to float within the flexible tubing 511 prior to the adhesive 517, and the multicore optical fiber 120 is free to float within the flexible tubing 511 after the adhesive 517, as well. Any compression (and axial strain) of the multicore optical fiber 120 responsive to an axial force F_{z} exerted on the distal end 515 of the elongated outer body 510 would therefore occur just proximally to the proximal to the location at which the multicore optical fiber 120 is fixed to the inner surface of the flexible tubing 511 by the adhesive 517. This compression (and axial strain) would be sensed through the force sensing region 540. Determination of the amount of axial force exerted on the distal end 515 involves measuring changes in the axial strain on the central optical fiber of the multicore optical fiber 120 at the force sensing region 540, as discussed above. The adhesive 517 may be an epoxy or an anaerobic adhesive material, for example, although different materials may be incorporated without departing from the scope of the present teachings.

FIG. 11 shows a similar concept as FIG. 10, but without the rigid tube 212. The multicore optical fiber 120 is fixed directly to the flexible tubing 511 in one location by the adhesive 517, and then any compression in the flexible tubing 511 will be transmitted to the fixed segment. Hence, the force sensing region 540 would occur proximally to the fixed section. Applying the adhesive 517 in a middle portion, for example, of a long elongated outer body 510 may be challenging, though. The fixed section defined by the adhesive 517 should be very small in comparison to the length of the elongated outer body 510, and placing the adhesive 517 involves the multicore optical fiber 120 being pushed through several centimeters of the flexible tubing 511. Alternative materials to the adhesive 517 may be, such as UV curable or heat curable glue, which would allow a smaller diameter elongated outer body 510 to be used. Accordingly, the multicore optical fiber 120 may be fixed to the flexible tubing 511 after it has been pushed through the flexible tubing 511. In other words, use of UV curable or heat curable glue, for example, enables external determination of the location(s) at which the multicore optical fiber 120 is fixed to the flexible tubing 511, even if the glue is located (but not cured) outside that location(s).

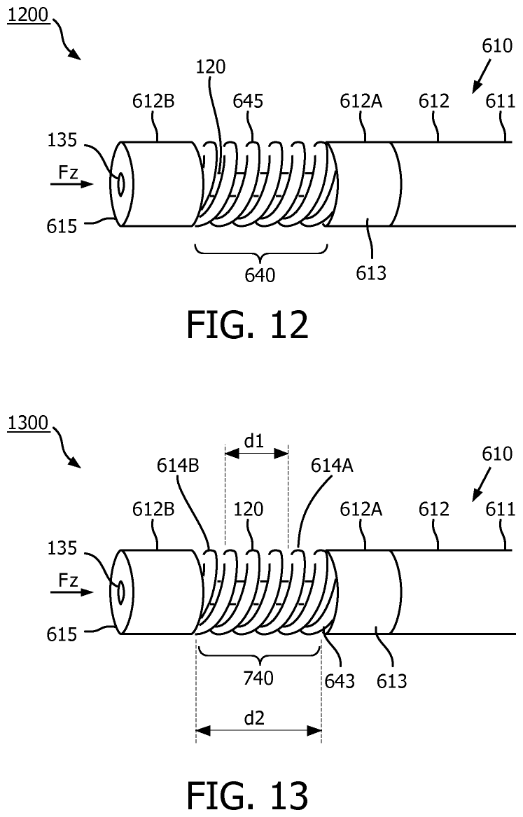

FIG. 12 is a plan view of an optical shape sensing device including a force sensing region having a coil spring, according to a representative embodiment.

Referring to FIG. 12, optical shape sensing device 1200 includes an elongated outer body 610, which includes flexible tubing 611 and rigid tube 612 attached to the flexible tubing 611. In the depicted embodiment, the rigid tube 612 is attached to a distal end 613 of the flexible tubing 611. The flexible tubing 611 enables the maneuvering of the optical shape sensing device 1200 through a passage, as discussed above. The optical shape sensing device 1200 also includes multicore optical fiber 120 extending longitudinally through the elongated outer body 610, and a termination piece (e.g., termination piece 130, not shown in FIG. 12) attached to a distal tip of the multicore optical fiber 120, as discussed above. The termination piece is positioned within the rigid tube 612, and includes the distal tip 135, which may substantially coincide with a distal end 615 of the elongated outer body 610. Shape sensing is enabled by the optical shape sensing device 1200 along the multicore optical fiber 120 clear to the distal tip 135 of the termination piece.

The optical shape sensing device 1200 further includes a force sensing region 640 integrated with the rigid tube 612 of the elongated outer body 610. The rigid tube 612 has a proximal rigid section 612A, a distal rigid section 612B, and a multithread coil spring 645 located in between, where the multicore optical fiber runs through the coil spring 645. In the depicted embodiment, the force sensing region 640 of the optical shape sensing device 1200 coincides with the coil spring segment 645, which is the elastic segment of the elongated outer body 610. That is, the coil spring 645 enables axial compression and expansion of the rigid tube 612 responsive to an axial force F_{z} exerted on the distal end 615 of the elongated body 610. Use of the coil spring 645 enables the elastic segment to be longer than other types of elastic segments, such as a pattern of slits (e.g., elastic segment 245) or a laser cut design.

The force sensing region 640, together with the processing unit 260 (not shown in FIG. 12), is configured to sense the amount of axial force F_{z} exerted on the distal end 615 of the elongated outer body 610, which corresponds to the distal end of the rigid tube 612. When the coil spring 645 compresses, the optical fiber 120 between the proximal and distal rigid sections 612A and 612B also compresses, and the axial strain in this area is used to calculate the applied force. The optical fiber 120 may be fixed to the proximal and distal rigid sections 612A and 612B using adhesive (not shown in FIG. 12), similar to the adhesive 217 discussed above. Determination of the amount of axial force exerted on the distal end 615 involves measuring changes in axial strain on the central optical fiber of the multicore optical fiber 120 at the force sensing region 640, as discussed above.

FIG. 13 is a simplified schematic diagram of a cut-away view of an optical shape sensing device including a force sensing region having a coil spring, according to a representative embodiment. Referring to FIG. 13, optical shape sensing device 1300 is substantially the same as the optical shape sensing device 1200, with the addition of proximal and distal rigid extensions 614A and 614B that extend within the coil spring 645 from the proximal and distal rigid sections 612A and 612B. Extending these solid parts (proximal and distal rigid extensions 614A and 614B) inside the coil spring 645 results in the axial strain induced in the multicore optical fiber 120 by the axial force F_{z} being larger than the axial strain induced in the coil spring 645.

More particularly, application of an axial force F_{z} results in a compression of the rigid tube 612 assembly indicated by δd. The axial strain over the length (d2) of the coil spring 645 is ε2=δd/d2, whereas the axial strain over the length (d1) of the exposed portion of the multicore optical fiber 120 (i.e., the space within the coil spring 645 between proximal and distal rigid extensions 614A and 614B) is ε1=δd/d1. Since d2>d1, it follows that ε1>ε2, which will result effectively in increased force sensitivity in the force sensing region 740 of the optical shape sensing device 1300, e.g., as compared to the force sensing region 640 of the optical shape sensing device 1200.

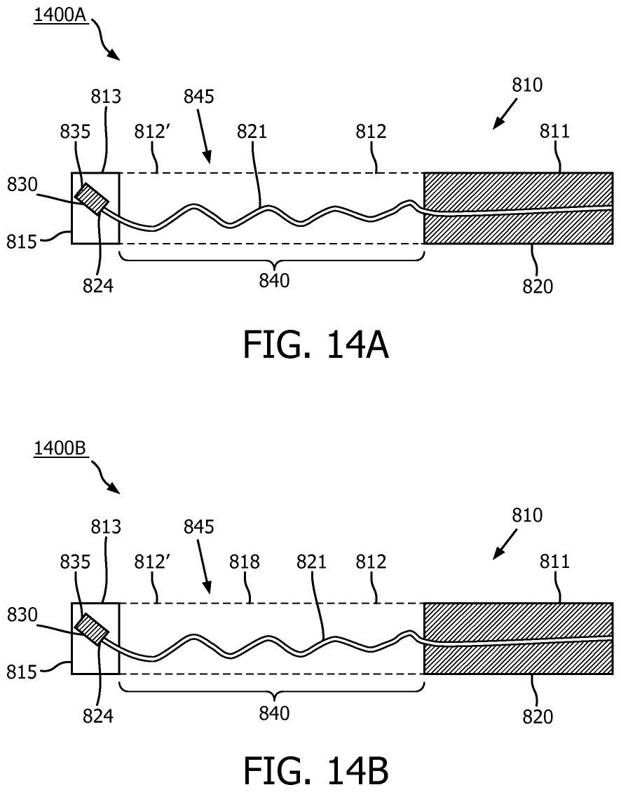

FIG. 14A is a simplified cross-sectional diagram of an optical shape sensing device including a force sensing region in which optical fiber has helical pattern, according to a representative embodiment.

Referring to FIG. 14A, optical shape sensing device 1400A includes an elongated outer body 810, which includes proximal flexible tubing 811, distal flexible tubing 812 attached to the proximal flexible tubing 811, and distal tube 813 attached to the distal flexible tubing 812. The proximal and distal flexible tubing 811 and 812 enable the maneuvering of the optical shape sensing device 1400A through a passage, as discussed above. The optical shape sensing device 1400A also includes multicore optical fiber 820 extending longitudinally through the elongated outer body 810, and a termination piece 830 attached to a distal tip 824 of the multicore optical fiber 820, as discussed above. The termination piece 830 is located within the distal tube 813 and includes a distal tip 835, which may substantially coincide with a distal end 815 of the elongated outer body 810. Shape sensing is enabled by the optical shape sensing device 1400 along the multicore optical fiber 820 to the distal tip 835 of the termination piece 830. The composition of the multicore optical fiber 820 is substantially the same as the multicore optical fiber 120, discussed above.

In the depicted embodiment, the multicore optical fiber 820 includes a helical portion 821 having a helical pattern. The helical portion 821 is embedded in compliant material 812′ within the distal flexible tubing 812, which increases axial sensitivity in multiple directions over other embodiments in which the multicore optical fiber has no helical patter. The helical portion 821 defines a deformation region 845, and the force sensing region 840 of the optical shape sensing device 1400 coincides with the deformation region 845. The complaint material 812′ may be silicon (Si), for example, although other materials with similar compliant properties may be incorporated without departing from the scope of the present teachings. Incorporation of the helical portion 821 engages multiple modes of deformation to provide higher resolution force-from-strain sensing.

The deformation region 845 enables axial compression and expansion of the distal flexible tubing 812 (and the compliant material 812′ therein) of the elongated outer body 810 responsive to an axial force F_{z} exerted on the distal end 815 of the elongated body 810. The force sensing region 840, together with the processing unit 260 (not shown in FIG. 14), is configured to sense the amount of axial force exerted on the distal end 815 of the elongated outer body 810. When the deformation region 845 compresses, the helical portion 821 of the multicore optical fiber 820 deforms in a manner reflected by the compliant material 812′, and thus captured by the force sensing region 840. Due to freedom of movement of the helical portion 821 within the compliant material 812′, forces in directions other than an axial direction may be detected via the force sensing region 840.

FIG. 14B is a simplified cross-sectional diagram of an optical shape sensing device including a force sensing region in which optical fiber has helical pattern, according to a representative embodiment. Referring to FIG. 14B, optical shape sensing device 1400B is substantially the same as the optical shape sensing device 1400A, with the addition of stiffening members 818, formed along the distal flexible tubing 812 to increase lateral stiffness. The stiffening members 818 may be formed of any lightweight, substantially rigid material, such as titanium, polyether ether ketone, polypropylene, nylon, polyimide, acetal, or acrylonitrile butadiene styrene, for example. Also, the stiffening members 818 may be arranged on an outer surface of the distal flexible tubing 812, as shown, or between the distal flexible tubing 812 and the compliant material 812′, although other arrangements of the distal flexible tubing 812 may be incorporated without departing from the scope of the present teachings.

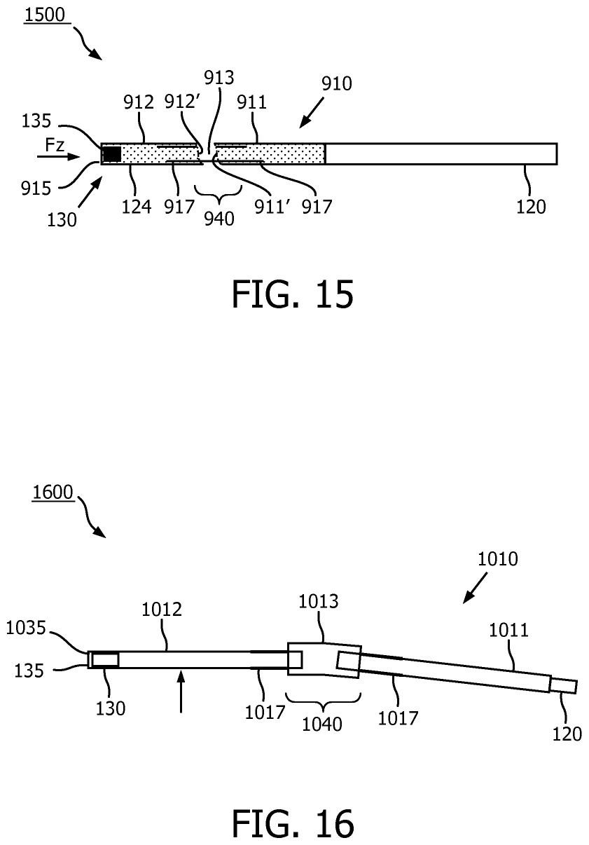

FIG. 15 is a simplified cross-sectional diagram of an optical shape sensing device including a force sensing region for sensing torsion, according to a representative embodiment.

Referring to FIG. 15, optical shape sensing device 1500 includes an elongated outer body 910 configured to maneuver through a passage, as discussed above. The elongated outer body 910 includes a proximal (first) substantially rigid portion 911 and a distal (second) substantially rigid portion 912, separated by a space 913 between the proximal and distal substantially rigid portions 911 and 912. The proximal and distal substantially rigid portions 911 and 912 may be formed of the same material(s) as the rigid tube 212, for example, discussed above with reference to FIG. 8.

The optical shape sensing device 1500 also includes multicore optical fiber 120 extending longitudinally through the elongated outer body 910, and a termination piece 130 attached to the distal tip 124 of the multicore optical fiber 120, as discussed above. The termination piece 130 is positioned within the distal substantially rigid portion 92, and includes the distal tip 135, which may substantially coincide with a distal end 915 of the elongated outer body 910. Shape sensing is enabled by the optical shape sensing device 1500 along the multicore optical fiber 120 to the distal tip 135 of the termination piece 130.

Adhesive 917 binds the multicore optical fiber 120 to portions of the inner surfaces of the proximal substantially rigid portion 911 and the distal substantially rigid portion 912, respectively, adjacent the space 913. The adhesive 917 prevents the multicore optical fiber 120 from sliding within the proximal and distal substantially rigid portions 911 and 912. The adhesive 917 may be an epoxy or an anaerobic adhesive material, for example, although different materials may be incorporated without departing from the scope of the present teachings.

In the depicted embodiment, the proximal substantially rigid portion 911 has a first angled edge 911′ and the distal substantially rigid portion 912 has a second angled edge 912′ complementary to the first angled edge 911′. The first and second angled edges 911′ and 912′ face one another across the space 913, and are shaped so that, when the elongated outer body 910 is compressed, the first and second angled edges 911′ and 912′ rotate with respect to one another, causing the multicore optical fiber 120 (adhered to the inner surfaces of the proximal and distal substantially rigid portions 911 and 912) to twist within the space 913. A force sensing region 940, which substantially coincides with the space 913, is configured to sense the amount of twisting (torsion) of the multicore optical fiber 120 in response to the axial force F_{z} exerted on the distal end 915 of the elongated body 910. Generally, the twisting of the multicore optical fiber 120 causes the at least two additional optical fibers, helically wrapped around the central optical fiber of the multicore optical fiber 120, to unravel or tighten to an extent proportional to the amount of axial force being exerted on the distal end 915. Thus, in an embodiment, the extent of unraveling or tightening may be used to determine the axial force F_{z}.

The force sensing region 940, together with the processing unit 260 (not shown in FIG. 15), is configured to sense the amount of axial force exerted on the distal end 915 of the elongated outer body 910, which corresponds to the distal end of the distal substantially rigid portion 912. When the proximal and distal substantially rigid portions 911 and 912 rotate with respect to one another, the multicore optical fiber 120 twists, the amount of twisting is used by the processing unit 260 to calculate the applied axial force, in accordance with a predetermined algorithm. Determination of torsion is described, for example, in U.S. Pat. No. 8,773,650 to Froggatt et al. (Jul. 8, 2014), and in U.S. Pat. No. 7,772,541 to Froggatt et al. (Aug. 10, 2010), both of which are hereby incorporated by reference in their entireties.

FIG. 16 is a simplified cross-sectional diagram of an optical shape sensing device including a force sensing region for sensing buckling of the optical shape sensing device, according to a representative embodiment.

Referring to FIG. 16, optical shape sensing device 1600 includes an elongated outer body 1010 configured to maneuver through a passage, as discussed above. The elongated outer body 1010 includes a proximal (first) substantially rigid portion 1011 and a distal (second) substantially rigid portion 1012, and flexible tubing 1013 connected between the proximal and distal substantially rigid portions 1011 and 1012. The flexible tubing 1013 enables the proximal and distal substantially rigid portions 1011 and 1012 to move relative to one another, enabling bending or buckling of the elongated outer body 1010. The proximal and distal substantially rigid portions 1011 and 1012 may be formed of the same material(s) as the rigid tube 212, for example, and the flexible tubing 1013 may by formed of the same material(s) as the flexible tubing 211, for example, discussed above with reference to FIG. 8.

The optical shape sensing device 1600 also includes multicore optical fiber 120 extending longitudinally through the elongated outer body 1010, and a termination piece 130 attached to the distal tip 124 of the multicore optical fiber 120, as discussed above. The termination piece 130 is positioned within the distal substantially rigid portion 1012, and includes the distal tip 135, which may substantially coincide with a distal end 1015 of the elongated outer body 1010. Shape sensing is enabled by the optical shape sensing device 1600 along the multicore optical fiber 120 to the distal tip 135 of the termination piece 130.

The optical shape sensing device 1600 further includes a force sensing region 1040 integrated with the elongated outer body 1010. More particularly, the force sensing region 1040 substantially coincides with a bendable portion of the flexible tubing 1013 (e.g., where there is no overlap between the flexible tubing 1013 and either of the proximal substantially rigid portion 1011 or the distal substantially rigid portion 1012). The force sensing region 1040 is configured to sense an axial force exerted F_{z} on the distal end 1015 of the elongated body 1010 based on determining an amount of buckling experienced by the flexible tubing 1013 and sensed by the force sensing region 1040 in response to the axial force F_{z}. That is, the force sensing region 1040 senses the axial force F_{z} via changes in curvature of the multicore optical fiber 120, or strain on the multicore optical fiber 120, within the flexible tubing 1013 resulting from buckling.

Adhesive 1017 binds the multicore optical fiber 120 to portions of the inner surfaces of the proximal substantially rigid portion 1011 and the distal substantially rigid portion 1012, respectively, adjacent the flexible tubing 1013. The adhesive 1017 prevents the multicore optical fiber 120 from sliding within the proximal and distal substantially rigid portions 1011 and 1012 to enable a more accurate determination of buckling caused by application of the axial force F_{z}. The adhesive 1017 may be an epoxy or an anaerobic adhesive material, for example, although different materials may be incorporated without departing from the scope of the present teachings.

The force sensing region 1040, together with the processing unit 260 (not shown in FIG. 16), is configured to sense the amount of axial force exerted on the distal end 1015 of the elongated outer body 210, which corresponds to the distal end of the distal substantially rigid portion 1012. When the flexible tubing 1013 buckles, the bare multicore optical fiber 120 also buckles, and the amount (or degree) of buckling is used by the processing unit 260 to calculate the applied axial force, in accordance with a predetermined algorithm. Buckling may be sensed, for example, through a change in the curvature of the multicore optical fiber. The greater the amount of buckling, the greater the curvature change. A calibration procedure may be used to model force as a function of curvature. Determination of curvature and changes thereto is described, for example, in U.S. Pat. No. 8,773,650 to Froggatt et al. (Jul. 8, 2014), and in U.S. Pat. No. 7,772,541 to Froggatt et al. (Aug. 10, 2010), both of which are hereby incorporated by reference in their entireties.

In other embodiments, the design of the outer surface of a conventional optical shape sensing device (e.g., a guidewire or catheter shaft) may be modified. For example, conventional guidewires and catheters may be made of nitinol, which is “braided,” and then coated with different types of materials (e.g., soft and flexible or more rigid). That is, the entire outer surface or outer body of the optical sensing device may be braided in the same (conventional) manner, but the material covering the braided design may differ in flexibility in various sections, depending on anticipated functionality, respectively. Alternatively, or in addition, construction the braided design may differ in various sections to change flexibility. That is, the conventional braided design may still be used in the majority of the optical sensing device, while a relatively small section the nitinol may be formed into a spring-like design that compresses in response to applied axial forces.

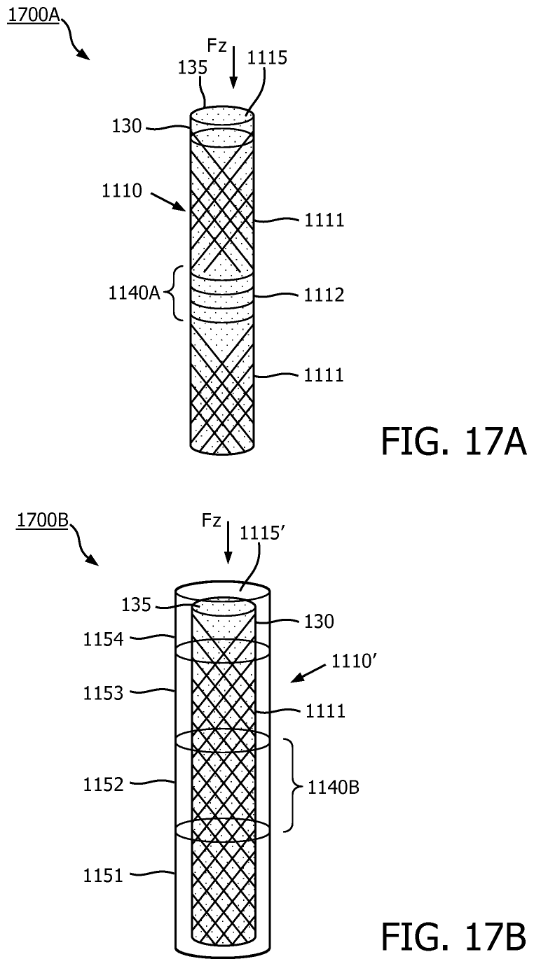

FIG. 17A is a simplified transparent plan view of an optical shape sensing device including a force sensing region, according to a representative embodiment.

Referring to FIG. 17A, optical shape sensing device 1700A includes an elongated outer body 1110, which includes braided design portions 1111 and a spring design portion 1112 formed integrally with and between the braided design portions 1111. The spring design portion 1112 compresses in response to applied axial forces, such as axial force F_{z}.

A multicore optical fiber (not shown) runs longitudinally through the elongated outer body 1110, and is fixed to the braided design portions 1111, e.g., using adhesive, on either end of the spring design portion 1112. A termination piece 130 is attached to a distal tip of the multicore optical fiber, and includes a distal tip 135, which may substantially coincide with a distal end 1115 of the elongated outer body 1110. A force sensing region 1140A of the optical shape sensing device 1700 substantially coincides with the spring design portion 1112. The force sensing region 1140, together with the processing unit 260 (not shown in FIG. 17A), is configured to determine the amount of axial force exerted on a distal end 1115 of the elongated outer body 1110 by sensing compression of the spring design portion 1112 responsive to the axial force F_{z}.

FIG. 17B is a simplified transparent plan view of an optical shape sensing device including a force sensing region, according to a representative embodiment.

Referring to FIG. 17B, optical shape sensing device 1700B includes an elongated outer body 1110′, which includes a braided design portion 1111 along substantially the entire length (i.e., there is not spring design portion). Rather, an elastic segment of the elongated outer body 1110′ is provided by use of different materials covering the braided design portion 1111. In the depicted embodiment, the elongated outer body 1110′ is covered by a first material in first material segment 1151, a second material in second material segment 1152, a third material in third material segment 1153, and a fourth material in fourth material segment 1154. The first and third materials, which may be the same, are rigid or substantially rigid materials, and the third material is a standard material for covering a termination piece (e.g., termination piece 130), such as standard PTFE, for example. The second material covering the second material segment 1152 is an elastic material, such as silicone or any biocompatible rubber-like material, for example. Accordingly, the second material segment 1152 compresses in response to applied axial forces, such as axial force F_{z}.

A multicore optical fiber (not shown) runs longitudinally through the elongated outer body 1110′, and is fixed to at least the first and third material segments 1151 and 1153, e.g., using adhesive, on either side of the end of the second material segment 1152. A termination piece 130 is attached to a distal tip of the multicore optical fiber, and includes a distal tip 135, which may substantially coincide with a distal end 1115′ of the elongated outer body 1110′. A force sensing region 1140B of the optical shape sensing device 1700B substantially coincides with the second material segment 1152. The force sensing region 1140B, together with the processing unit 260 (not shown in FIG. 17B), is configured to determine the amount of axial force exerted on a distal end 1115′ of the elongated outer body 1110′ by sensing compression of the second material segment 1152 responsive to the axial force F_{z}.

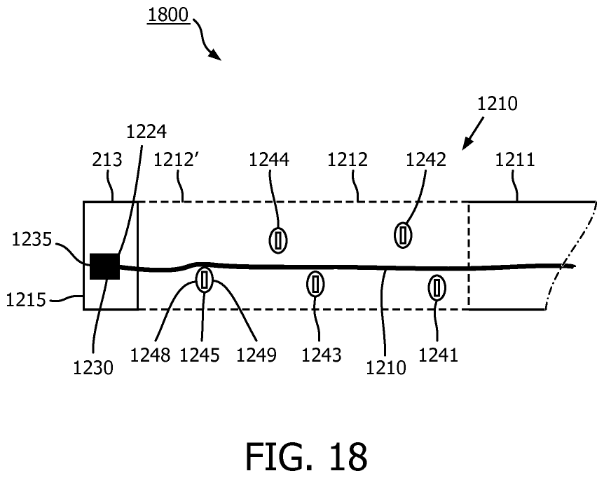

FIG. 18 is a simplified cross-sectional diagram of an optical shape sensing device including multiple force sensing regions embedded in compliant material, according to a representative embodiment.

Referring to FIG. 18, optical shape sensing device 1800 includes an elongated outer body 1210, which includes proximal flexible tubing 1211, distal flexible tubing 1212 attached to the proximal flexible tubing 1211, and distal tube 1213 attached to the distal flexible tubing 1212. The proximal and distal flexible tubing 1211 and 1212 enable the maneuvering of the optical shape sensing device 1800 through a passage, as discussed above. The optical shape sensing device 1800 also includes multicore optical fiber 1220 extending longitudinally through the elongated outer body 1210, and a termination piece 1230 attached to a distal tip 1224 of the multicore optical fiber 1220, as discussed above. In the depicted embodiment, a portion of the multicore optical fiber 1220 is embedded in compliant material 1212′ within the distal flexible tubing 1212. The termination piece 1230 is located within the distal tube 1213, and includes a distal tip 1235, which may substantially coincide with a distal end 1215 of the elongated outer body 1210. Shape sensing is enabled by the optical shape sensing device 1800 along the multicore optical fiber 1220 to the distal tip 1235 of the termination piece 1230. The composition of the multicore optical fiber 1220 is substantially the same as the multicore optical fiber 120, discussed above.

The optical shape sensing device 1800 further includes multiple force sensing regions 1241, 1242, 1243, 1244 and 1245 embedded in the compliant material 1212′, surrounding the multicore optical fiber 1220. Each of the force sensing regions 1241 to 1245 includes a solid element 1248 inside a corresponding perforation 1249 through the distal flexible tubing 1212 and the compliant material 1212′. The solid element 1248 may be a metal bead, for example, and the compliant material 1212′ may be silicon (Si), for example, although other compliant materials with similar properties, respectively, may be incorporated, without departing from the scope of the present teachings.

The force of a contact on the termination piece 1230 and/or the distal flexible tubing 1212 (axial or lateral) pushes one or more of the solid elements 1248 inside the distal flexible tubing 1212. This changes the position of the one or more solid elements 1248, and thus the shape of the compliant material 1212′, creating a small change in the shape of the optical shape sensing device 1800 corresponding to the contact point. In the example depicted in FIG. 18, a substantial lateral force (not shown) has displaced at least the solid element 1248 of the force sensing region 1245, such that it is in contact with the multicore optical fiber 1220 (changing the shape of the multicore optical fiber 1220, as well as the shape of the compliant material 1212′). The extent of the displacement is sensed by at least the force sensing region 1245 (and possibly one or more of the other force sensing regions 1241-1244). Therefore, the force sensing regions 124-1245, together with the processing unit 260 (not shown in FIG. 18), are configured to sense the amount of lateral forces, as well as axial force, exerted on the termination piece 1230 and/or the distal flexible tubing 1212.

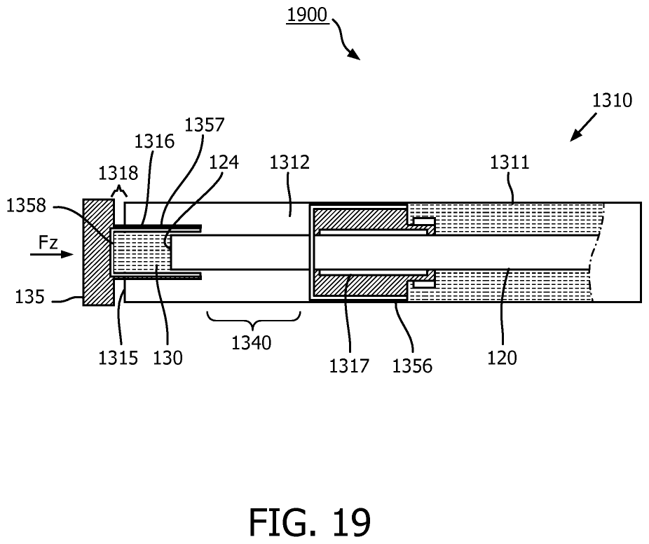

FIG. 19 is a simplified schematic diagram of a cut-away view of an optical shape sensing device including a force sensing region and a stopper, according to a representative embodiment.

Referring to FIG. 19, optical shape sensing device 1900 includes an elongated outer body 1310, which includes flexible tubing 1311 and substantially rigid tube 1312 attached to the flexible tubing 1311. In the depicted embodiment, the rigid tube 1312 is attached to a distal end 1313 of the flexible tubing 1311, and may have varying degrees of rigidity, although the rigid tube 1312 is less flexible than the flexible tubing 1311. The flexible tubing 1311 enables the maneuvering of the optical shape sensing device 1900 through a passage, as discussed above. The optical shape sensing device 1900 further includes a disk 1358 attached to distal inner tubing 1357, which extends into a distal side of the rigid tube 1312 through a distal end 1315 of the elongated outer body 1310. In an uncompressed state, the disk 1358 is spaced apart from the distal end 1315 (which may also be referred to as a stopper) by gap 1318, as shown in FIG. 19.

The optical shape sensing device 1900 also includes multicore optical fiber 120 extending longitudinally through the elongated outer body 1310, and termination piece 130 attached to the distal tip 124 of the multicore optical fiber 120, as discussed above. The termination piece 130 is positioned within the rigid tube 1312, and has a distal tip 135. More particularly, the termination piece 130 and at least a portion of the multicore optical fiber 120 are positioned within the distal inner tubing 1357, which is inside the rigid tube 1312. The termination piece 130 and the at least a portion of the multicore optical fiber 120 are bound to the inside surface of the distal inner tubing 1357 using adhesive 1316. In the uncompressed stated, the distal tip 135 (inside the distal inner tubing 1357) extends beyond the distal end 1315 (stopper) of the elongated outer body 1310, as discussed below. Shape sensing is enabled by the optical shape sensing device 1900 along the multicore optical fiber 120 clear to the distal tip 135 of the termination piece 130.

A force sensing region 1340 is integrated with the elongated outer body 1310 in the rigid tube 1357. In an embodiment, the force sensing region 1340 is located between a proximal end of the distal inner tubing 1357 and a distal end of additional inner tubing 1356 located at a proximal side of the rigid tube 1312. A portion of the multicore optical fiber 120 extends through the additional inner tubing 1356, and is bound to an inner surface of the additional inner tubing 1356 by adhesive 1317. Thus, the force sensing region 1340 is effectively defined by an area between the proximal end of the distal inner tubing 1357 and the distal end of the additional inner tubing 1356. This focuses axial compression and expansion in the force sensing region 1340 within the defined area responsive to an axial force F_{z} exerted on the disk 1358. The adhesive 1316 and 1317 may be an epoxy or an anaerobic adhesive material, for example, although different materials may be incorporated without departing from the scope of the present teachings.

Accordingly, when the axial force F_{z} is exerted on the disk 1358, the rigid tube 1312 and the multicore optical fiber 120 compress within the force sensing region 120, and the gap 1318 becomes smaller (closes). Depending on the magnitude of the axial force F_{z}, the compression continues until the gap 1318 closes completely, that is, the disk 1358 is in physical contact with the distal end 1315. Thus, the size of the gap 1318 limits the amount of axial force (and the extent of compression of the force sensing region 1340) exerted on the termination piece 130 and the multicore optical fiber 120, thereby protecting the multicore optical fiber 120 from breakage in the force sensing region 1340 or elsewhere. The gap size may be selected based on mechanical properties of the multicore optical fiber 120 and the termination piece 130, as well as the maximum amount of force a user wants to detect.

In addition, the force sensing region 1340, together with the processing unit 260 (not shown in FIG. 19), are configured to sense the compression and determine the amount of axial force exerted on the disk 1358 and/or the distal end 1315 of the elongated outer body 1310. The axial strain in the area of the force sensing region 1340 is used to calculate the applied force. Determination of the amount of axial force exerted on the disk 1358 and/or the distal end 1315 involves measuring changes in axial strain on the central optical fiber of the multicore optical fiber 120, as discussed above.

While various embodiments have been illustrated and described in detail in the drawings and foregoing description, such illustration and description are to be considered illustrative or exemplary and not restrictive; the invention is not limited to the disclosed embodiments.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1-15. (canceled)

16. A system for registering a device with a stored three-dimensional (3D) representation of a region of interest, the device comprising an outer body for maneuvering through a passage in the region of interest and a force sensing region integrated with the outer body, the device being a shape sensing or having at least one electromagnetic (EM) sensor, insitu ultrasound sensing, dielectric sensing, the system comprising a computing device and a computer-readable storage medium storing instructions executable by the computing device to:
determine a plurality of points at which an end of the outer body contacts a surface of an object in the region of interest, based on forces exerted on the end when contacting the surface and detected by the force sensing region and received by the system; and
register the determined plurality of points with points in the 3D representation of the region of interest so that the registered points are in a common space.

17. The system of claim 16, wherein said computer-readable storage medium stores instructions executable by the computing device to implement said registration by at least:
determining sets of 3D coordinates of the determined plurality of points in a shape space; and
registering the sets of 3D coordinates to the points in the 3D representation, instructions using a registration algorithm.

18. The system of claim 17, wherein said registration algorithm comprises a deformable Iterative Closest Point (ICP) algorithm.

19. The system of claim 16, wherein the stored 3D representation of the region of interest comprises an x-ray image, an MR image, a CT image, a cone beam CT (CBCT) image, a positron emission tomography (PET) scan image, an ultrasound image or an optical image.

20. The system of claim 16, wherein the stored 3D representation of the region of interest comprises a segmented surface model.

21. The system of claim 16, wherein the stored 3D representation of the region of interest comprises a known signature that is enabled to affect shape data of the passage in the region of interest.

22. The system of claim 21, wherein the known signature comprises at least one of thermal signature or defined curvature signature.

23. The system of claim 21, wherein the known signature comprises a profile of navigation signatures derived from at least one previous procedure involving shape sensing navigation of the passage in the region of interest.

24. The system of claim 16, wherein said computer-readable storage medium storing instructions is executable by the computing device to further:
determine stiffness of the passage at the plurality of points at which the distal end of the outer body contacts the inner surface of the passage, based on axial forces exerted on the distal end, as measured by the force sensing region and received by the system,
wherein said stored executable instructions are further adapted to implement said registration of the determined plurality of points with points in the 3D representation of the region to include incorporating indications of stiffness for each the registered points in the common space.

25. The system of claim 16, wherein said computer-readable storage medium storing instructions is executable by the computing device to further:
indicate a position of the device when navigating through the passage using the registered points.

26. The system of claim 16, wherein said computer-readable storage medium storing instructions is executable by the computing device to further:
define a planned path in the 3D representation of the region of interest, the planned path substantially corresponding to the passage;
at an initial times, register the initial position of the device and the planned path in a 3D shape space, and determine an initial transformation using the registration in the 3D shape space to transform the initial position of the device to a 3D region space of the 3D representation of the region of interest;
at subsequent times, while continuing to navigate the device through the passage, apply the transformation to the device to iteratively transform subsequent positions of the device, determined using the at least one EM sensor if the device comprises such an EM sensor, corresponding to the subsequent times from the 3D shape space into the 3D region space;
determine a best overlapping region between the planned path and the subsequent positions of the device in the 3D region space, the best overlapping region comprising a portion of the 3D representation of the region of interest in which the subsequent positions of the device most closely coincide with the planned path;
register the subsequent positions of the device only in the best overlapping region and the planned path only in the best overlapping region in the 3D space shape, and determining an updated transformation algorithm using the subsequent registration in the best overlapping region; and
apply the updated transformation algorithm to the device to again transform the subsequent positions of the device, determined using the at least one EM sensor if the device is said interventional device, from the 3D shape space into the 3D region space.

27. A method for registering a shape sensing device with a three-dimensional (3D) representation of a region of interest, the shape sensing device comprising an outer body for maneuvering through a passage in the region of interest and a force sensing region integrated with the outer body, the method comprising:
determining a plurality of points at which an end of the outer body contacts a surface of an object in the region of interest, based on forces exerted on the end when contacting the surface and detected by the force sensing region; and
registering the determined plurality of points with points in the 3D representation of the region of interest so that the registered points are in a common space.

28. The method of claim 27, wherein the shape sensing device is an optical shape sensing (OSS) device, and wherein determining the plurality of points comprises determining a plurality of positions of the distal end of the outer body using optical shape sensing when the distal end contacts the inner surface of the passage, the determined plurality of positions corresponding to the plurality of points.

29. The method of claim 27, wherein the shape sensing device comprises a guidewire or a catheter.

30. A method for registering a device with a previously obtained three-dimensional (3D) representation of a region of interest, the device comprising an elongated outer body for maneuvering through a passage in the region of interest, the device being a shape sensing device or an interventional device having at least one electromagnetic (EM) sensor attached to a distal end of the elongated outer body, the method comprising:
defining a planned path in the 3D representation of the region of interest, the planned path substantially corresponding to the passage;
inserting the device in the passage to begin navigating the device through the passage at an initial position;
at an initial times, registering the initial position of the device and the planned path in a 3D shape space, and determining an initial transformation algorithm using the registration in the 3D shape space to transform the initial position of the device to a 3D region space of the 3D representation of the region of interest;
at subsequent times, while continuing to navigate the device through the passage, applying the transformation algorithm to the device to iteratively transform subsequent positions of the device, determined using the at least one EM sensor if the device is said interventional device, corresponding to the subsequent times from the 3D shape space into the 3D region space;
determining a best overlapping region between the planned path and the subsequent positions of the device in the 3D region space, the best overlapping region comprising a portion of the 3D representation of the region of interest in which the subsequent positions of the device most closely coincide with the planned path;
registering the subsequent positions of the device only in the best overlapping region and the planned path only in the best overlapping region in the 3D space shape, and determining an updated transformation algorithm using the subsequent registration in the best overlapping region; and
applying the updated transformation algorithm to the device to again transform the subsequent positions of the device, determined using the at least one EM sensor if the device is said interventional device, from the 3D shape space into the 3D region space.
